(19) United States
(12) Patent Application Publication
(10) Pub. No.: US 2026/0137646 A1
(43) Pub. Date: May 21, 2026

(54) PENTADECANOYLCARNITINE FOR TREATMENT OF CONDITIONS RELATED TO THE QUALITY OF AGING AND LONGEVITY

(71) Applicant: EPITRACKER, INC., San Diego, CA (US)

(72) Inventor: Stephanie Venn-Watson, San Diego, CA (US)

(21) Appl. No.: 19/249,920

(22) Filed: Jun. 25, 2025

Related U.S. Application Data
(63) Continuation of application No. 17/816,950, filed on Aug. 2, 2022, now Pat. No. 12,370,166.
(60) Provisional application No. 63/275,353, filed on Nov. 3, 2021.
(60) Provisional application No. 63/340,710, filed on May 11, 2022.

(51) Int. Cl. A61K 31/23; A61K 31/20; A61P 9/00; A61P 11/06; A61P 13/12; A61P 17/06; A61P 25/16; A61P 25/24; A61P 37/06; A61P 37/08

(52) U.S. Cl. CPC A61K 31/23; A61K 31/20; A61P 9/00; A61P 11/06; A61P 13/12; A61P 17/06; A61P 25/16; A61P 25/24; A61P 37/06; A61P 37/08

(57) ABSTRACT

Administration of pentadecanoylcarnitine or pentadecanoic acid is provided for prevention, management or treatment of aggression, allergies, allergic rhinitis, Alzheimer's disease, anxiety and anxiety disorders, amyotrophic lateral sclerosis, arthritis, asthma, atherosclerosis, attention-deficit hyperactivity disorder, bipoloar disorder, brain damage, cancer, cardiovascular disease, cholestatic pruritis, depression, chronic obstructive pulmonary disease (COPD), cocaine abuse, cough, dermatitis, depression, drug-seeking behavior, gastrointestinal disorders, facial erythema associated with rosacea, glaucoma, hepatic diseases, hyperactive bladder, hypersensitivity disorders, hypertension, impulsivity, inflammation, mental disorders and conditions, metabolic disorders, migraines, nasal and sinus congestion, nausea, neuropathic pain with and symptoms of multiple sclerosis, neurological and neuropsychiatric disorders, obesity, obsessive-compulsive disorders, opioid-induced respiratory depression, osteoarthritis, pain, Parkinson disease, pathological gambling, peptic ulcers, schizophrenia, sleep disorders, spinal cord injury, tardive dyskinesia, tics and behavioral problems with Tourette's syndrome; as well as for supporting appetite, cardiovascular health, hematologic health, memory, metabolic health, mood, prolonged REM sleep, renal health, sexuality, sociability and metabolic, hematological, renal, and weight loss.

### CROSS-REFERENCE TO RELATED APPLICATIONS

This application is a continuation of U.S. application Ser. No. 17/816,950, filed Aug. 2, 2022, which claims the benefit of U.S. Provisional Application No. 63/340,710, filed May 11, 2022, and U.S. Provisional Application No. 63/275,353, filed Nov. 3, 2021. Each of the aforementioned applications is incorporated by reference herein in its entirety, and is hereby expressly made a part of this specification.

### STATEMENT REGARDING FEDERALLY SPONSORED R&D

The United States Government has certain rights in this invention, under ONR SBIR Agreement No. N00014-21-9-0002.

### FIELD OF THE INVENTION

Administration of pentadecanoylcarnitine or pentadecanoic acid is provided for prevention, management or treatment of aggression, allergies, allergic rhinitis, Alzheimer's disease, anxiety and anxiety disorders, amyotrophic lateral sclerosis, arthritis, asthma, atherosclerosis, attention-deficit hyperactivity disorder, bipolar disorder, brain damage, cancer, cardiovascular disease, cholestatic pruritis, depression, chronic obstructive pulmonary disease (COPD), cocaine abuse, cough, dermatitis, depression, drug-seeking behavior, gastrointestinal disorders, facial erythema associated with rosacea, glaucoma, hepatic diseases, hyperactive bladder, hypersensitivity disorders, hypertension, impulsivity, inflammation, mental disorders and conditions, metabolic disorders, migraines, nasal and sinus congestion, nausea, neuropathic pain with and symptoms of multiple sclerosis, neurological and neuropsychiatric disorders, obesity, obsessive-compulsive disorders, opioid-induced respiratory depression, osteoarthritis, pain, Parkinson disease, pathological gambling, peptic ulcers, schizophrenia, sleep disorders, spinal cord injury, tardive dyskinesia, tics and behavioral problems with Tourette's syndrome; as well as for supporting appetite, cardiovascular health, hematologic health, memory, metabolic health, mood, prolonged REM sleep, renal health, sexuality, sociability and metabolic, hematological, renal, and weight loss.

### BACKGROUND OF THE INVENTION

Carnitine is an amino acid present in food, primarily in animal sources, but it can also be made endogenously. L-carnitine is the bioavailable and biologically active isomer. Carnitines are almost entirely found within cells, where they can shuttle long-chain fatty acids into mitochondria as long-chain acylcarnitines to enable β-oxidation-generated energy for cellular activities.

### SUMMARY OF THE INVENTION

Compositions and methods are provided for prevention, management or treatment of aggression, allergies, allergic rhinitis, Alzheimer's disease, anxiety and anxiety disorders, amyotrophic lateral sclerosis, arthritis, asthma, atherosclerosis, attention-deficit hyperactivity disorder, bipolar disorder, brain damage, cancer, cardiovascular disease, cholestatic pruritis, depression, chronic obstructive pulmonary disease (COPD), cocaine abuse, cough, dermatitis, depression, drug-seeking behavior, gastrointestinal disorders, facial erythema associated with rosacea, glaucoma, hepatic diseases, hyperactive bladder, hypersensitivity disorders, hypertension, impulsivity, inflammation, mental disorders and conditions, metabolic disorders, migraines, nasal and sinus congestion, nausea, neuropathic pain with and symptoms of multiple sclerosis, neurological and neuropsychiatric disorders, obesity, obsessive-compulsive disorders, opioid-induced respiratory depression, osteoarthritis, pain, Parkinson disease, pathological gambling, peptic ulcers, schizophrenia, sleep disorders, spinal cord injury, tardive dyskinesia, tics and behavioral problems with Tourette's syndrome; as well as for supporting appetite, cardiovascular health, hematologic health, memory, metabolic health, mood, prolonged REM sleep, renal health, sexuality, sociability and metabolic, hematological, renal, and weight loss. These compositions comprise pentadecanoylcarnitine or pentadecanoic acid, or salts thereof, which may be administered in combination with other medicaments or as part of various treatment regimens as described herein. The provided compositions are effective for modulating markers as described herein. Methods are provided for administering the compositions.

Accordingly, in a generally applicable first aspect (i.e., independently combinable with any of the aspects or embodiments identified herein), a pharmaceutical composition is provided comprising: pentadecanoylcarnitine or pentadecanoic acid; and a pharmaceutically acceptable carrier.

In an embodiment of the first aspect (i.e., independently combinable with any of the aspects or embodiments identified herein), the composition is in a unit dosage form.

In an embodiment of the first aspect (i.e., independently combinable with any of the aspects or embodiments identified herein), the pharmaceutical composition is configured for administration of from 2.5 mg to 50 mg, per 1 kg of body weight, of the pentadecanoylcarnitine or pentadecanoic acid, or pharmaceutically acceptable salts thereof to a patient.

In an embodiment of the first aspect (i.e., independently combinable with any of the aspects or embodiments identified herein), the pharmaceutical composition is configured for administration once per day.

In an embodiment of the first aspect (i.e., independently combinable with any of the aspects or embodiments identified herein), the pharmaceutical composition comprises from 0.01 mg to 10000 mg of pentadecanoylcarnitine or pentadecanoic acid or pharmaceutically acceptable salts thereof.

In a generally applicable second aspect (i.e., independently combinable with any of the aspects or embodiments identified herein), use is provided of a pharmaceutical composition of the first aspect or any embodiment thereof, in the manufacture of a medicament for treatment or prophylaxis of conditions as described herein.

In an embodiment of the second aspect (i.e., independently combinable with any of the aspects or embodiments identified herein), the use is in the manufacture of a medicament for treatment or prophylaxis of conditions as described herein.

In an embodiment of the second aspect (i.e., independently combinable with any of the aspects or embodiments identified herein), the pharmaceutical composition is configured to modulate a marker of aging-associated conditions described herein or a symptom of conditions described herein.

In an embodiment of the second aspect (i.e., independently combinable with any of the aspects or embodiments identified herein), the marker of conditions related to an indication herein is selected from the group consisting of serum or plasma pentadecanoylcarnitine concentration, red blood cell indices (i.e. hemoglobin, red blood cells), serum or plasma cholesterol, triglycerides, insulin, glucose, gamma-glutamyl transpeptidase, ferritin, or iron.

In a generally applicable third aspect (i.e., independently combinable with any of the aspects or embodiments identified herein), the pharmaceutical composition is configured to increase a serum, red blood cell, or tissue concentration of pentadecanoylcarnitine to between 0.2 μM and 20 μM.

In an embodiment of the third aspect (i.e., independently combinable with any of the aspects or embodiments identified herein), the pentadecanoylcarnitine or pharmaceutically acceptable salts thereof is provided as a pharmaceutical composition in a unit dosage form.

In an embodiment of the third aspect (i.e., independently combinable with any of the aspects or embodiments identified herein), the unit dosage form comprises from 0.01 mg to 10000 mg of the pentadecanoylcarnitine.

In an embodiment of the third aspect (i.e., independently combinable with any of the aspects or embodiments identified herein), the pharmaceutical composition further comprises a plurality of different small molecule metabolites described herein.

In an embodiment of the third aspect (i.e., independently combinable with any of the aspects or embodiments identified herein), from 2.5 mg to 50 mg of pentadecanoylcarnitine or pharmaceutically acceptable salts thereof is administered to the patient, per 1 kg of body weight, per day.

In an embodiment of the third aspect (i.e., independently combinable with any of the aspects or embodiments identified herein), the pentadecanoylcarnitine or pharmaceutically acceptable salts thereof is administered to the patient once per day.

In an embodiment of the third aspect (i.e., independently combinable with any of the aspects or embodiments identified herein), a serum, tissue, or a red blood cell membrane concentration of pentadecanoylcarnitine is increased 1.25 to 6 times above the patient's baseline levels to achieve concentrations between 0.5 μM and 20 μM.

In a generally applicable fourth aspect (i.e., independently combinable with any of the aspects or embodiments identified herein), use is provided of pentadecanoylcarnitine or pentadecanoic acid to achieve body concentrations of pentadecanoylcarnitine of from 1 μM and 20 μM to prevent, manage or treat aggression, allergies, allergic rhinitis, Alzheimer's disease, anxiety and anxiety disorders, amyotrophic lateral sclerosis, arthritis, asthma, atherosclerosis, attention-deficit hyperactivity disorder, bipolar disorder, brain damage, cancer, cardiovascular disease, cholestatic pruritis, depression, chronic obstructive pulmonary disease (COPD), cocaine abuse, cough, dermatitis, depression, drug-seeking behavior, gastrointestinal disorders, facial erythema associated with rosacea, glaucoma, hepatic diseases, hyperactive bladder, hypersensitivity disorders, hypertension, impulsivity, inflammation, mental disorders and conditions, metabolic disorders, migraines, nasal and sinus congestion, nausea, neuropathic pain with and symptoms of multiple sclerosis, neurological and neuropsychiatric disorders, obesity, obsessive-compulsive disorders, opioid-induced respiratory depression, osteoarthritis, pain, Parkinson disease, pathological gambling, peptic ulcers, schizophrenia, sleep disorders, spinal cord injury, tardive dyskinesia, tics and behavioral problems with Tourette's syndrome; as well as supporting appetite, cardiovascular health, hematologic health, memory, metabolic health, mood, prolonged REM sleep, renal health, sexuality, sociability and metabolic, hematological, renal, and weight loss.

In a generally applicable fifth aspect (i.e., independently combinable with any of the aspects or embodiments identified herein), a composition substantially as described herein is provided.

In a generally applicable sixth aspect (i.e., independently combinable with any of the aspects or embodiments identified herein), a composition substantially as described herein is provided.

In a generally applicable seventh aspect (i.e., independently combinable with any of the aspects or embodiments identified herein), a use substantially as described herein is provided.

### DESCRIPTION OF THE DRAWINGS

FIG. 1A shows increased serum pentadecanoic acid (C15:0) concentrations within 1 month on the modified fish diet that was sustained through 6 months.

FIG. 1B shows increased pentadecanoylcarnitine concentrations within 1 month on the modified fish diet that was sustained through 6 months.

### DETAILED DESCRIPTION

Compositions including pentadecanoylcarnitine or pentadecanoic acid, and associated methods for treatment of conditions including aggression, allergies, allergic rhinitis, Alzheimer's disease, anxiety and anxiety disorders, amyotrophic lateral sclerosis, arthritis, asthma, atherosclerosis, attention-deficit hyperactivity disorder, bipolar disorder, brain damage, cancer, cardiovascular disease, cholestatic pruritis, depression, chronic obstructive pulmonary disease (COPD), cocaine abuse, cough, dermatitis, depression, drug-seeking behavior, gastrointestinal disorders, facial erythema associated with rosacea, glaucoma, hepatic diseases, hyperactive bladder, hypersensitivity disorders, hypertension, impulsivity, inflammation, mental disorders and conditions, metabolic disorders, migraines, nasal and sinus congestion, nausea, neuropathic pain with and symptoms of multiple sclerosis, neurological and neuropsychiatric disorders, obesity, obsessive-compulsive disorders, opioid-induced respiratory depression, osteoarthritis, pain, Parkinson disease, pathological gambling, peptic ulcers, schizophrenia, sleep disorders, spinal cord injury, tardive dyskinesia, tics and behavioral problems with Tourette's syndrome; as well as supporting appetite, cardiovascular health, hematologic health, memory, metabolic health, mood, prolonged REM sleep, renal health, sexuality, sociability and metabolic, hematological, renal, and weight loss are provided.

Aging is associated with chronic, low-grade inflammation characterized by increased circulating levels of proinflammatory cytokines, neutrophils, and progressively activated macrophages. This pro-inflammatory state is a significant risk factor for both morbidity and mortality in the elderly people (Franceschi C, Campisi J (2014) Chronic inflammation (inflammaging) and its potential contribution to age-associated diseases. J Gerontol Ser A 69:S4-S9). People who live at least 100 years are less likely to have developed a proinflammatory state with age, further supporting that chronic, low-grade inflammation impairs quality and duration of life (Vasto S, Candore G, Balistreri M, Colonna-Romano G, Grimaldi M P, Listi F et al. (2007) Inflammatory networks in ageing, age-related diseases, and longevity. Mech Ageing and Dev 128:83-91). As such, interventions to reduce inflammation of aging have been proposed to concurrently treat multiple aging-associated diseases, resulting in improved quality of life and expanded longevity.

As people age, the prevalence of anemia can increase dramatically. Comparisons of populations aged 40, 60, 80, 90 and 100 (total n=1,980) had the following prevalence of anemia: 16.1%, 19.1%, 41.1%, 46.2%, and 57.1% (Zhai Y, Yin Z X, Xu J W, Liu Y Z, Shi X M (2010) Anemia status and its relevant factors among elderly people aged above 80 years old in longevity areas in China. Chinese J of Prev Med 44:115-118). The presence of anemia and low hemoglobin concentration increased the 3-year mortality risk by 25% and decreased longevity among older people (Lyu Y, Yin Z, Luo J, Shi X, Zeng Y (2015) Zhonghua Liuxingbingxue Zazhi 36:682-686). Means to prevent and treat anemia of aging are expected to improve quality of life and expand longevity.

Elevated cholesterol with aging can negatively impact quality of life or longevity. (Kriesberg R A and Kasim S (1987) Cholesterol metabolism and aging, Am J Med 82:54-60). Elevated cholesterol, especially elevated low-density lipoprotein (LDL) cholesterol, have been identified as underlying causes of or contributors to cardiovascular disease, including atherosclerosis, which also increase in prevalence with age. Lower cholesterol levels, especially for people under 50 years old, have been associated with improved longevity (Anderson K M, Castelli W P, Levy D (1987) Cholesterol and mortality: 30 years of follow-up from the Framingham Study JAMA 257:2176-2180). Similarly, triglyceride levels can be major predicting factors for human longevity, with lower triglyceride levels present in long-lived families compared to controls (Vaarhorst A A M, Beekman M, Suchiman E H D, van Heemst D V, Houwing-Duistermaat J J, Westemdorp R G et al (2010) Lipid metabolism in long-lived families: the Leiden Longevity Study. AGE 33:219-227). Preventing and treating dyslipidemia have been highlighted as important to improve quality of life and expand longevity.

Prediabetic and diabetic conditions, including hyperglycemia and insulin resistance, result in impaired quality of life and longevity. From 1999 to 2011, the average number of years lost from diabetes has increased by 46% in men and 44% in women (Gregg E W, Zhuo X, Cheng Y J, Albright A L, Narayan K M V, Thompson T J (2014) Trends in lifetime risk and years of life lost due to diabetes in the USA, 1985-2011: A modelling study. Lancet Diab Endocrinol 2:867-874). Insulin resistance increases with advanced age, and people who live over one hundred years have lower insulin resistance compared to those who were younger (Paolisso G, Barbieri M, Rizzo M R, Carella C, Rotondi M, Bonafe M et al (2001) Low insulin resistance and preserved 3-cell function contribute to human longevity but are not associated with TH-INS genes. Exp Gerontol 37:147-156). Treatments long used to treat type 2 diabetes, including metformin, have been demonstrated to expand longevity (Novelle M G, Ali A, Dieguez C, Bernier M, de Cabo R (2016) Metformin: A hopeful promise in aging research. CSH Perspectives 6:a025932), and there is an active effort to discover other compounds that may help treat hyperglycemia and insulin resistance and expand longevity.

The prevalence of chronic liver disease and accompanying cirrhosis and hepatocellular carcinoma have been increasing at an alarming rate, especially in developed countries. This increase is due primarily to nonalcoholic fatty liver disease (NAFLD) associated with the global rise in obesity and metabolic syndrome (including elevated glucose, dyslipidemia, and insulin resistance). Chronic liver disease contributes to morbidity and mortality, and by aiming to decrease liver failure, transplants, and cancer, therapeutics for liver disease can improve quality of life and expand longevity (Lim Y S, Kim W R (2008) The global impact of hepatic fibrosis and end-stage liver disease. Clinics in Liver Dis 12:733-746).

Iron overload and hyperferritinemia with aging can negatively impact quality of life or longevity. Iron accumulates with age in tissues, including the brain (Hirose W, Ikematsu K, and Tsuda R (2003) Age-associated increases in heme oxygenase-1 and ferritin immunoreactivity in the autopsied brain. Legal Med 5:S360-366). Because iron induces oxidative damage to tissues, resulting in age-related diseases, such as Alzheimer's disease, compounds that reduce iron overload and hyperferritinemia have been proposed as therapeutic targets for aging-associated diseases (Bartzokis G, Tishler T A, Lu P H, Villablanca P, Altshuler L L, Carter M et al. (2007) Brain ferritin iron may influence age- and gender-related risks of neurodegeneration. Neurobiol Aging 28:414-423).

Aging skin and poor wound healing can negatively impact quality of life. Aging skin has changes to structure and function that impairs its integrity and ability to heal (Farage M A, Miller K W, Elsner P, Maibach H I (2013) Characteristics of the aging skin. Adv Wound Care 2: doi: 10.1089). Compounds that prevent or correct intrinsic changes with age that influence skin integrity and repair can aid in improving the quality of life.

With respect to aging and pain, over half of people over 65 years old report to have bothersome pain, most of which have pain in multiple sites and have at least two chronic medical conditions such as arthritis, cardiometabolic diseases, and obesity (Patel K V et al. (2013) Prevalence and impact of pain among older adults in the United States: Findings from the 2011 National Health and Aging Trends Study. Pain 154:2649-2657). As such, anti-inflammatory agents, analgesics, and compounds that attenuate cardiometabolic diseases may aid in alleviating pain, including pain associated with aging. As an example, compounds that reduce prostaglandin E2 (PGE2) can aid in reducing inflammation, pain, and fever, including pain caused by osteoarthritis (Lee A S et al. (2013) A current review of molecular mechanisms regarding osteoarthritis and pain. Gene 527:440-447). Other compounds that reduce inflammation associated with autoimmune diseases, such as rheumatoid arthritis, can attenuate joint inflammation and pain. As an example, interleukin-17A (IL-17a) is an important contributor to autoimmune diseases, including multiple sclerosis and rheumatoid arthritis, and a compound that lowers IL-17A may help to alleviate pain from rheumatoid arthritis (Iwakura Y et al. (2008) The roles of IL-17A inflammatory immune responses and host defense against pathogens. Immunol Rev 226). Further, compounds that reduce chronic systemic inflammation associated with aging and cardiometabolic diseases may attenuate these diseases and their associated pain.

With respect to aging and allergies, allergies are one of the fastest growing health problems in people aged over 15 years, and 5% to 10% of allergies are affecting elderly people (Martinis M D et al. (2017) Allergy and aging: an old/new emerging health issue. Aging and Dis 8:162-175). Elderly can be at higher risk of allergies due to physiological changes with aging, concurrent diseases, polydrug therapy, and compromised systems, including the dermal, gastrointestinal, and respiratory systems. Interleukin-17A (IL-17a) is an important contributor to allergic responses, including systemic and dermal hypersensitivities and allergic airway inflammation (Iwakura Y et al. (2008) The roles of IL-17A inflammatory immune responses and host defense against pathogens. Immunol Rev 226). As such, compounds that reduce allergy responses, including reduction of IL-17A, can improve the quality of life for people, including the elderly.

With respect to aging and sleep disorders, sleep problems have been reported to affect up to 40% of the elderly population (Vitiello M V (1997) Sleep disorders and aging: understanding the causes. J Gerontol 52A:M189-M191). This high prevalence is attributed, in part, to physiological changes with aging, as well as the presence of chronic disease. Control of chronic conditions, such as pain, have been demonstrated to improve the quality of sleep and resolve insomnia (Monjan A and Foley D (1996) Incidence of chronic insomnia associated with medical and psychosocial factors: an epidemiologic study among older persons. Sleep Res 25:108). Despite growing concern over the use of benzodiazapines and other sedatives, especially among the elderly, people over 60 years old are more likely to receive sedative prescriptions compare to people aged 40 to 59 (Baum C et al. (1986) Drug utilization in the U.S.—1985: Seventh annual review. Rockville, MD: Food and Drug Administration, Center for Drugs and Biologies). As such, use of natural compounds that may help alleviate underlying medical conditions impacting sleep can be used as a first line means of improving the quality of sleep and life. As a potential alternative to sedatives, PGE2 inhibitors, which can be used to reduce inflammation, pain and fevers, may also aid in stemming PGE2's role in stimulating the wake centers near the posterior hypothalamus (Hayaishi O (1991) Molecular mechanisms of sleep-wake regulation: roles of prostaglandins D2 and E2. FASEB J 5:2575-2581).

With respect to aging and gastrointestinal and/or digestive disorders, aging impacts normal digestion, resulting in disorders associated with sensation, inflammation, poor swallowing, imbalanced microbiota, malabsorption, and malnutrition (Shamburek R D and Farrar J T (1990) Disorders of the digestive system in the elderly. New Engl J Med 322:438-443). People older than 65 years represent 25% of all inflammatory bowel disease hospitalizations, and older age was a significant risk factor for increased mortality and more severe gastrointestinal disease compared to younger patients (Ananthakrishnan A N et al. (2009) Inflammatory bowel disease in the elderly is associated with worse outcomes: a national study of hospitalizations. Inflamm Bowel Dis 15: 182-289). Compounds that lower gastrointestinal inflammation, restore a balanced microbiota and/or improve proper absorption of nutrients may aid in alleviating digestive disorders. Interleukin-17A (IL-17A) is a contributor to chronic intestinal inflammation, and lowering IL-17A secretion may aid in alleviating digestive disorders (Coccia M et al. (2012) IL-1β mediates chronic intestinal inflammation by promoting the accumulation of IL-17A secreting innate lymphoid cells and CD4+ Th17 cells. J Exp Med 209:1595).

With respect to aging and skin conditions and/or wound healing, advanced age can impair wound healing (Sgnoc R and Gruber J (2013) Age-related aspects of cutaneous wound healing: a mini review. Gerontol 59:159-164). This impairment can be due to chronic inflammatory conditions present with age, including increases in cytokines, such as IL-6, and chemokines, such as CXCL8 (also called interleukin 8 (IL-8)), which is elevated in psoriasis. There are some differences present in fetal and elderly wound healing, however, that may be beneficial. As an example, fetal and elderly wounds heal with no to little scarring, respectively. This scarless repair may be due to lower levels of decorin and IL-8, an extracellular matrix proteoglycan and chemokine, respectively, observed in fetal tissue (Beanes S R et al. (2001) Down-regulation of decorin, a transforming growth factor-beta modulator, is associated with scarless fetal wound healing. J Pediatr Surg 36:1666-71; Liechty K W (1997) Diminished interleukin-8 (IL-8) production in the fetal wound healing response. J Surg Res 77:80-84). As such, lower levels of IL-6, IL-8, and decorin may help stem inflammation associated with chronic, non-healing wounds and enable this healing to occur with minimal scarring.

Carnitine is an amino acid present in food, primarily in animal sources, but it can also be made endogenously (Kendler B S. Carnitine: an overview of its role in preventive medicine. Prev Med 15:373-390 (1986)). L-carnitine is the bioavailable and biologically active isomer (Rebouche C J. Kinetics, pharmacokinetics, and regulation of L-carnitine and acetyl-L-carnitine metabolism. Ann NY Acad Sci 1033:30-41 (2004)), and supplementation with L-carnitine has proposed health benefits related to neurological disorders, cardiovascular disease, and obesity (Flanagan, J. L., Simmons, P. A., Vehige, J. Wilcox, M. D. P., Garrett, Q. Role of carnitine in disease. Nutr Metabol 7:30 (2010), Indiveri, C., Iacobazzi, V. Tonazzi, A., Giangregorio, N., Infantino, V. Convertini, P. et al. The mitochondrial carnitine/acylcarnitine carrier: function, structure and physiopathology. Mol Asp Med 32:223-233 (2011)).

Carnitines are almost entirely found within cells, where they can shuttle long-chain fatty acids into mitochondria as long-chain acylcarnitines to enable β-oxidation-generated energy for cellular activities (Flanagan, J. L., Simmons, P. A., Vehige, J. Wilcox, M. D. P., Garrett, Q. Role of carnitine in disease. Nutr Metabol 7:30 (2010)). Higher circulating concentrations of long-chain acylcarnitines have been associated with higher risks of age-related diseases, including inflammation, mitochondrial dysfunction, cardiovascular disease, type 2 diabetes, and osteoarthritis (Jarrell, Z. R., Smith, M. R., Hu, X., Orr, M., Liu, K. H., Quyyumi, A. A. et al. Plasma acylcarnitine levels increase with healthy aging. Aging 12:13555-13570 (2020), Kalim, S., Clish, C. B., Wenger, J., Elmariah, S., Yeh, R. W., et al. A plasma long-chain acylcarnitine predicts cardiovascular morality in incident dialysis patients. J Am Heart Assoc 2:e000542 (2013), Koh, A. S., Gao, F., Liu, J., Fridianto, K. T., Ching, J., Tan, R. S. et al. Metabolomic profile of arterial stiffness in aged adults. Diab Vase Dis Res 15:74-80 (2018), Kendler B S. Carnitine: an overview of its role in preventive medicine. Prev Med 15:373-390 (1986), Bouchouirab et al. Plasma Palmitoyl-Carnitine (AC16:0) Is a Marker of Increased Postprandial Nonesterified Incomplete Fatty Acid Oxidation Rate in Adults With Type 2 Diabetes, Can J Diabetes 2018 August; 42(4):382-388.el. doi: 10.1016/j.jcjd.2017.09.002. Epub 2017 Nov. 9., Tootsi, K., Kals, J., Zilmer, M., Paapstel, K., Ottas, A., Martson, A. Medium- and long-chain acylcarnitines are associated with osteoarthritis severity and arterial stiffness in end-stage osteoarthritis patients: a case-control study. Int J Rheum Dis 21:1211-1218 (2018)).

While long-chain acylcarnitines have been shown to increase with age, short and medium odd-chain acylcarnitines, specifically containing C5:0, C7:0 and C9:0, decrease with age; this decrease with age was not noted with long odd-chain acylcarnitines, such as C15:0 and C17:0 (Jarrell et al. Plasma acylcarnitine levels increase with healthy aging, Aging (Albany NY). 2020 Jul. 15; 12(13): 13555-13570).

Pentadecanoic acid (C15:0) is a long, odd-chain saturated fatty acid present in trace levels in dairy fat, as well as some types of plants and fish (Jenkins, B. et al. A review of odd-chain fatty acid metabolism and the role of pentadecanoic acid (C15:0) and heptadecanoic acid (C17:0) in health and disease. Molecules 20:2425-2444 (2015)). Large, prospective cohort studies have repeatedly shown that higher blood concentrations of pentadecanoic acid are associated with lower risks of developing chronic diseases, including type 2 diabetes, cardiovascular disease, and heart failure (Forouhi, N. G. et al. Differences in the prospective association between individual plasma phospholipid saturated fatty acids and incident type 2 diabetes: the EPIC-InterAct case-cohort study. Lancet Diab Endocrinol 14:70146-9 (2014), Trieu, K. et al. Biomarkers of dairy fat intake, incident cardiovascular disease, and all-cause mortality: A cohort study, systematic review, and meta-analysis. PLoS Med 18:e1003763 (2021), Djousse, L. et al. Serum individual nonesterified fatty acids and risk of heart failure in older adults. Cardiology 146:351-358 (2021)). Higher dietary intake and circulating concentrations of pentadecanoic acid have also been associated with lower mortality and greater longevity (Zhuang, P., Cheng, L., Wang, J. & Zhang Y. Saturated fatty acid intake is associated with total mortality in a nationwide cohort study. J Nutrition 149:68-77 (2019), Trieu et al. 2021 Biomarkers of dairy fat intake, incident cardiovascular disease, and all-cause mortality: A cohort study, systematic review, and meta-analysis, PLOS Medicine, Published: Sep. 21, 2021 https://doi.org/10.1371/joumal.pmed.1003763, Manca, C., Carta, G, Murru, E., Abolghasemi A, et al. Circulating fatty acids and endocannabiniodome-related mediator profiles associated to human longevity. GeroSci https://doi.org/10.1007/s11357-021-00342-0 (2021)).

Pentadecanoic acid is a dual, partial peroxisome proliferator-activated receptor (PPAR) α/δ agonist and AMP-activated protein kinase (AMPK) activator with demonstrated ability to repair mitochondrial function, improve the stability of red blood cell membranes, and decrease proliferation of breast cancer cells (Venn-Watson, S., Lumpkin, R., Dennis, E. A. Efficacy of dietary odd-chain saturated fatty acid pentadecanoic acid parallels broad associated health benefits in humans: could it be essential?Sci Rep 10:8161 (2020), Fu, W. C. et al. Pentadecanoic acid promotes basal and insulin-stimulated glucose uptake in C2C12 myotubes. Food Nutr Res 65:10.29219/fnr.v65.4527 (2021), To, N. B., Nguyen, Y. T., Moon, J. Y., Ediriweera, M. K., Cho, S. K. Pentadecanoic acid, an odd-chain fatty acid, suppresses the stemness of MCF-7/SC human breast cancer stem-like cells through JAK2/STAT3 signaling. Nutrients 12:1663 (2020)). In human cell systems mimicking various disease states, pentadecanoic acid has both anti-inflammatory and antifibrotic properties (Venn-Watson, S., Lumpkin, R., Dennis, E. A. Efficacy of dietary odd-chain saturated fatty acid pentadecanoic acid parallels broad associated health benefits in humans: could it be essential?Sci Rep 10:8161 (2020)). Further, diet-induced obese mice modeling type 2 diabetes were supplemented daily with oral pentadecanoic acid for approximately 12 weeks and demonstrated lower glucose, cholesterol, body weight gain, and pro-inflammatory cytokines compared to non-supplemented controls (Venn-Watson, S., Lumpkin, R., Dennis, E. A. Efficacy of dietary odd-chain saturated fatty acid pentadecanoic acid parallels broad associated health benefits in humans: could it be essential?Sci Rep 10:8161 (2020)).

Daily pentadecanoic acid supplementation over 11 weeks also lowered inflammation, cholesterol, and triglycerides and attenuated anemia and liver fibrosis in an in vivo model of nonalcoholic fatty liver disease (Venn-Watson, S., Lumpkin, R., Dennis, E. A. Efficacy of dietary odd-chain saturated fatty acid pentadecanoic acid parallels broad associated health benefits in humans: could it be essential?Sci Rep 10:8161 (2020)). Because pentadecanoic acid 1) is an established dietary active fatty acid not readily made by the body, 2) has lower body levels repeatedly associated with poorer cardiometabolic and liver health, and 3) has demonstrated beneficial and pleiotropic activities directly related to cardiometabolic and liver health, pentadecanoic acid has been proposed as an essential fatty acid (Venn-Watson, S., Lumpkin, R., Dennis, E. A. Efficacy of dietary odd-chain saturated fatty acid pentadecanoic acid parallels broad associated health benefits in humans: could it be essential?Sci Rep 10:8161 (2020)).

Bottlenose dolphins (Tursiops truncatus) are long-lived, large-brained mammals that develop chronic aging-associated conditions similar to humans, including metabolic syndrome, chronic inflammation, nonalcoholic fatty liver disease, and Alzheimer's disease (Gunn-Moore, D., Kaidanovich-Beilin, O., Iradi, M. C. G., Gunn-Moore, F., Lovestone, S. Alzheimer's disease in humans and other animals: A consequence of post reproductive life span and longevity rather than aging. Alz Dementia 14: 195-204 (2018), Venn-Watson, S., Smith, C. R., Gomez, F., Jensen, E. D. Physiology of aging among healthy, older bottlenose dolphins (Tursiops truncatus): comparisons with aging humans. J Comp Physiol 181:667-680 (2011), Venn-Watson, S., Benham, C., Carlin, K., DeRienzo, D., St. Leger, J. Hemochromatosis and fatty liver disease: building evidence for insulin resistance in bottlenose dolphins (Tursiops truncatus). J Zoo Wildlf Med 43:10.1638 (2012), Venn-Watson, S., Smith, C. R., Stevenson, S., Parry, C., Daniels, R. Jensen, E., et al. Blood-based indicators of insulin resistance and metabolic syndrome in bottlenose dolphins (Tursiops truncatus). Front Endocrinol:10.3389 (2013)). Similar to humans, dolphins with higher circulating odd-chain saturated fatty acid concentrations have a lower risk of metabolic syndrome and liver disease; feeding dolphins a modified diet with fish containing higher odd-chain saturated fatty levels, including C15:0 and C17:0, resulted in a shifted serum metabolome, lower insulin and cholesterol, as well as attenuated anemia (Venn-Watson, S., Baird, M., Novick, B., Parry, C., Jensen, E. D. Modified fish diet shifted serum metabolome and alleviated chronic anemia in bottlenose dolphins (Tursiops truncatus): Potential role of odd-chain saturated fatty acids. PLoS ONE doi10.1371/journal.pone.0230769 (2020)).

It is an object of certain of the embodiments to provide a composition comprising pentadecanoylcarnitine or pentadecanoic acid for use in achieving a body concentration of pentadecanoylcarnitine in the subject from about 1 μM to 20 μM. Body concentration can include but is not limited to any in vivo concentration, including blood concentration, plasma concentration, or other bodily fluid concentration. It is an object of certain of the embodiments to provide a composition comprising pentadecanoylcarnitine or pentadecanoic acid for use in treatment of one or more conditions selected from aggression, allergies, allergic rhinitis, Alzheimer's disease, anxiety and anxiety disorders, amyotrophic lateral sclerosis, arthritis, asthma, atherosclerosis, attention-deficit hyperactivity disorder, bipolar disorder, brain damage, cancer, cardiovascular disease, cholestatic pruritis, depression, chronic obstructive pulmonary disease (COPD), cocaine abuse, cough, dermatitis, depression, drug-seeking behavior, gastrointestinal disorders, facial erythema associated with rosacea, glaucoma, hepatic diseases, hyperactive bladder, hypersensitivity disorders, hypertension, impulsivity, inflammation, mental disorders and conditions, metabolic disorders, migraines, nasal and sinus congestion, nausea, neuropathic pain with and symptoms of multiple sclerosis, neurological and neuropsychiatric disorders, obesity, obsessive-compulsive disorders, opioid-induced respiratory depression, osteoarthritis, pain, Parkinson disease, pathological gambling, peptic ulcers, schizophrenia, sleep disorders, spinal cord injury, tardive dyskinesia, tics and behavioral problems with Tourette's syndrome; as well as supporting appetite, cardiovascular health, hematologic health, memory, metabolic health, mood, prolonged REM sleep, renal health, sexuality, sociability and metabolic, hematological, renal, and weight loss.

It is an object of certain of the embodiments to provide a method for detecting protective factors for and risk factors against conditions provided herein, including but not limited to inflammation, anemia, hyperglycemia, dyslipidemia, hyperinsulinemia, liver disease, iron overload, impaired skin integrity, wound healing, scarring, pain, allergies, sleep disorders and problems, and gastrointestinal disorders and problems, and other related conditions in mammal subjects, such as companion animals and humans. An object of certain of the embodiments is to provide a method for treating conditions including but not limited to aging-associated conditions that impact quality of life or longevity in mammal subjects, such as companion animals and humans. An object of certain of the embodiments is to provide a method for detecting conditions including but not limited to aging-associated conditions that impact quality of life or longevity in mammal subjects, such as companion animals and humans. It is an object of certain of the embodiments to provide a method for increasing the serum, plasma, or erythrocyte membrane level of pentadecanoylcarnitine in mammal subjects, such as companion animals and humans. An object of certain of the embodiments is to provide a pentadecanoylcarnitine or pentadecanoic acid supplement or prescription therapeutic for treating or preventing conditions including but not limited to those disclosed herein. An object of certain of the embodiments is to provide a method for detecting and/or treating conditions as disclosed herein in mammal subjects, such as companion animals and humans, which is easy to accomplish in a cost-effective manner.

An object of certain of the embodiments is to provide a method for modulating markers of conditions as disclosed herein in mammal subjects, such as companion animals and humans. An object of certain of the embodiments is to provide a method for detecting conditions as disclosed herein in mammal subjects, such as companion animals and humans. An object of certain of the embodiments is to provide a method for treatment of conditions as disclosed herein in mammal subjects, such as companion animals and humans. An object of certain of the embodiments is to provide a method for prophylaxis of conditions as disclosed herein in mammal subjects, such as companion animals and humans. An object of certain of the embodiments is to provide a method for prophylaxis of a condition provided herein in mammal subjects, such as companion animals and humans.

An object of certain of the embodiments is to provide a method for increasing pentadecanoylcarnitine in the sera, plasma, or erythrocyte membranes of mammal subjects, such as companion animals and humans. An object of certain of the embodiments is to provide a method for detecting or treating conditions as disclosed herein in mammal subjects, such as companion animals and humans. An object of certain of the embodiments is to provide a pentadecanoylcarnitine supplement substantially free from other small molecule metabolites in mammal subjects, such as companion animals and humans.

It is an object of certain of the embodiments is to provide a method for detecting and treating conditions as disclosed herein in mammal subjects, such as companion animals and humans. An object of certain of the embodiments is to provide pentadecanoylcarnitine or pentadecanoic acid for treating conditions as disclosed herein in mammal subjects, such as companion animals and humans. An object of certain of the embodiments is to provide a method for prophylaxis of conditions as disclosed herein in mammal subjects, such as companion animals and humans. An object of certain of the embodiments is to provide a method for detecting or treating conditions as disclosed herein in mammal subjects, such as companion animals and humans. An object of certain of the embodiments is to provide pentadecanoylcarnitine as a supplement for treating conditions as disclosed herein, such as in companion animals and humans.

An object of certain of the embodiments is to provide a bioavailable form of pentadecanoylcarnitine or pentadecanoic acid to mammal subjects, such as companion animals and humans. An object of certain of the embodiments is to provide pentadecanoylcarnitine or pentadecanoic acid with one or more other small molecule biochemicals described herein to mammal subjects, such as companion animals and humans. An object of certain embodiments is to provide a method for increasing small pentadecanoylcarnitine or pentadecanoic acid in the sera of mammal subjects, such as companion animals and humans. An object of certain of the embodiments is to provide a method for altering concentrations of pentadecanoylcarnitine or pentadecanoic acid as described herein in the sera, plasma, or erythrocyte membranes of mammal subjects, such as companion animals and humans.

One or more than one of the aforementioned objects is provided by or achieved by the various compositions, methods, and uses as described herein.

### Definitions

The term “alcohol” as used herein is a broad term, and is to be given its ordinary and customary meaning to a person of ordinary skill in the art (and is not to be limited to a special or customized meaning), and refers without limitation to any compound as described herein incorporating one or more hydroxy groups, or being substituted by or functionalized to include one or more hydroxy groups.

The term “short-chain fatty acid” as used herein is a broad term, and is to be given its ordinary and customary meaning to a person of ordinary skill in the art (and is not to be limited to a special or customized meaning), and refers without limitation to a fatty acid with 2-6 carbon atoms

The term “medium-chain fatty acid” as used herein is a broad term, and is to be given its ordinary and customary meaning to a person of ordinary skill in the art (and is not to be limited to a special or customized meaning), and refers without limitation to a fatty acid with 7-12 carbon atoms.

The term “long-chain fatty acid” as used herein is a broad term, and is to be given its ordinary and customary meaning to a person of ordinary skill in the art (and is not to be limited to a special or customized meaning), and refers without limitation to a fatty acid with 13-22 carbon atoms.

The term “very long chain fatty acid” as used herein is a broad term, and is to be given its ordinary and customary meaning to a person of ordinary skill in the art (and is not to be limited to a special or customized meaning), and refers without limitation to a fatty acid with 23 or more carbon atoms.

The term “derivative” as used herein is a broad term, and is to be given its ordinary and customary meaning to a person of ordinary skill in the art (and is not to be limited to a special or customized meaning), and refers without limitation to any compound as described herein incorporating one or more derivative groups, or being substituted by or functionalized to include one or more derivative groups. Derivatives include but are not limited to esters, amides, anhydrides, acid halides, thioesters, phosphates, triphosphates, and β-sulfenyl derivatives.

The term “hydrocarbon” as used herein is a broad term, and is to be given its ordinary and customary meaning to a person of ordinary skill in the art (and is not to be limited to a special or customized meaning), and refers without limitation to any moiety comprising only carbon and hydrogen atoms. A functionalized or substituted hydrocarbon moiety has one or more substituents as described elsewhere herein.

The term “lipid” as used herein is a broad term, and is to be given its ordinary and customary meaning to a person of ordinary skill in the art (and is not to be limited to a special or customized meaning), and refers without limitation to saturated and unsaturated oils and waxes, derivatives, amides, glycerides, small molecule metabolites, fatty alcohols, sterol and sterol derivatives, phospholipids, ceramides, sphingolipids, tocopherols, and carotenoids, among others.

The terms “pharmaceutically acceptable” as used herein is a broad term, and is to be given its ordinary and customary meaning to a person of ordinary skill in the art (and is not to be limited to a special or customized meaning), and refers without limitation to those compounds, materials, compositions, and/or dosage forms which are, within the scope of sound medical judgment, suitable for contact with the tissues of and/or for consumption by human beings and animals without excessive toxicity, irritation, allergic response, or other problem complications commensurate with a reasonable risk/benefit ratio.

The terms “pharmaceutically acceptable salts” and “a pharmaceutically acceptable salt thereof” as used herein are broad terms, and are to be given their ordinary and customary meaning to a person of ordinary skill in the art (and is not to be limited to a special or customized meaning), and refer without limitation to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′-dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as, for example, The Merck Index. Any suitable constituent can be selected to make a salt of the therapeutic agents discussed herein, provided that it is non-toxic and does not substantially interfere with the desired activity. In addition to salts, pharmaceutically acceptable precursors and derivatives of the compounds can be employed. Pharmaceutically acceptable amides, lower alkyl derivatives, and protected derivatives can also be suitable for use in compositions and methods of preferred embodiments. While it may be possible to administer the compounds of the preferred embodiments in the form of pharmaceutically acceptable salts, it is generally preferred to administer the compounds in neutral form.

The term “pharmaceutical composition” as used herein is a broad term, and is to be given its ordinary and customary meaning to a person of ordinary skill in the art (and is not to be limited to a special or customized meaning), and refers without limitation to a mixture of one or more compounds disclosed herein with other chemical components, such as diluents or carriers. The pharmaceutical composition facilitates administration of the compound to an organism. Pharmaceutical compositions can also be obtained by reacting compounds with inorganic or organic acids or bases. Pharmaceutical compositions will generally be tailored to the specific intended route of administration.

The term “carrier” as used herein is a broad term, and is to be given its ordinary and customary meaning to a person of ordinary skill in the art (and is not to be limited to a special or customized meaning), and refers without limitation to a compound that facilitates the incorporation of a compound into cells or tissues. For example, without limitation, dimethyl sulfoxide (DMSO) is a commonly utilized carrier that facilitates the uptake of many organic compounds into cells or tissues of a subject. Water, saline solution, ethanol, and mineral oil are also carriers employed in certain pharmaceutical compositions.

The term “diluent” as used herein is a broad term, and is to be given its ordinary and customary meaning to a person of ordinary skill in the art (and is not to be limited to a special or customized meaning), and refers without limitation to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The term “excipient” as used herein is a broad term, and is to be given its ordinary and customary meaning to a person of ordinary skill in the art (and is not to be limited to a special or customized meaning), and refers without limitation to a substance that is added to a pharmaceutical composition to provide, without limitation, bulk, consistency, stability, binding ability, lubrication, disintegrating ability etc., to the composition. A “diluent” is a type of excipient.

The term “subject” as used herein is a broad term, and is to be given its ordinary and customary meaning to a person of ordinary skill in the art (and is not to be limited to a special or customized meaning), and refers without limitation to an animal that is the object of treatment, observation or experiment. “Animal” includes cold- and warm-blooded vertebrates and invertebrates such as fish, shellfish, reptiles and, in particular, mammals. “Mammal” includes, without limitation, dolphins, mice, rats, rabbits, guinea pigs, dogs, cats, sheep, goats, cows, horses, primates, such as monkeys, chimpanzees, and apes, and, in particular, humans. In some embodiments, the subject is human.

The terms “treating,” “treatment,” “therapeutic,” or “therapy” as used herein are broad terms, and are to be given their ordinary and customary meaning (and are not to be limited to a special or customized meaning) and, without limitation, do not necessarily mean total cure or abolition of the disease or condition. Any prophylaxis of any undesired markers of a disease or condition, undesired signs of a disease or condition, or undesired symptoms of a disease or condition, to any extent, can be considered treatment and/or therapy. Any alleviation, lessening, lowering, or mitigation of any undesired markers of a disease or condition, undesired signs of a disease or condition, or undesired symptoms of a disease or condition, to any extent, can be considered treatment and/or therapy. Any promoting, supporting, enhancement, boosting, elevating, or improvement of any desired markers, desired physical states, desired physical conditions, desired mental states, desired mental conditions, health states, or health conditions, either in itself, or in association with a disease or condition, to any extent, can be considered treatment and/or therapy. Any modulation of any markers of a disease or condition, a sign of a disease or condition, a symptom of a disease or condition, a physical state, a physical condition, a mental state, a mental condition, a health condition, a health state, an aging condition, or an aging state, either in itself, or in association with a disease or condition, to any extent, can be considered treatment and/or therapy. Modulation can include, but is not limited to, shifting a marker, value, level, or other indicator of a physical, mental, health, or aging state or condition from one indicative of a relatively less healthy state or condition to one indicative of a relatively more healthy state or condition; or shifting a marker, value, level, or other indicator of a physical, mental, or health condition from one associated with a certain risk of a disease or condition to one indicative of a relatively lower risk of a disease or condition; or producing a desired reduction in variability of a marker, value, level, or other indicator of a physical, mental, health, or aging state or condition. Furthermore, treatment may include acts that may worsen the patient's overall feeling of well-being or appearance.

The terms “therapeutically effective amount” and “effective amount” as used herein are broad terms, and are to be given its ordinary and customary meaning to a person of ordinary skill in the art (and are not to be limited to a special or customized meaning), and are used without limitation to indicate an amount of an active compound, or pharmaceutical agent, that elicits the biological or medicinal response indicated. For example, a therapeutically effective amount of compound can be the amount needed to prevent, alleviate or ameliorate markers or symptoms of a condition or prolong the survival of the subject being treated. This response may occur in a tissue, system, animal or human and includes alleviation of the signs or symptoms of the disease being treated. Determination of a therapeutically effective amount is well within the capability of those skilled in the art, in view of the disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

The term “solvents” as used herein is a broad term, and is to be given its ordinary and customary meaning to a person of ordinary skill in the art (and is not to be limited to a special or customized meaning), and refers without limitation to compounds with some characteristics of solvency for other compounds or means, that can be polar or nonpolar, linear or branched, cyclic or aliphatic, aromatic, naphthenic and that includes but is not limited to: alcohols, derivatives, diesters, ketones, acetates, terpenes, sulfoxides, glycols, paraffins, hydrocarbons, anhydrides, heterocyclics, among others.

Any percentages, ratios or other quantities referred to herein are on a weight basis, unless otherwise indicated.

### Small Molecule Metabolites

In addition to pentadecanoylcarnitine or pentadecanoic acid, other small molecule metabolites may be present in compositions or administered to subjects. Small molecule metabolites are low molecular weight (typically less than 900 daltons, but sometimes higher) and can include but are not limited to amino acids, peptides, carbohydrates, cofactors and vitamins, xenobiotics, or lipids (including monohydroxy fatty acids, medium chain fatty acids, long chain fatty acids, very long chain fatty acids, dicarboxylic fatty acids, phosphatidylcholines, phosphatidylethanolamines, lysophospholipids, plasmalogens, lysoplasmalogens, monoacylglycerols, diacylglycerols, sphingomyelins, or ceramides) that can be identified and measured in the body and as provided herein. Small molecule metabolites can originate from ingestion of food or other oral products or produced endogenously. Small molecule metabolites are referred to and described using conventional nomenclature as is employed by one of skill in the art.

When specific compounds or classes of compounds are referred to herein, these compounds or classes of compounds may be produced by metabolic processes (e.g., by administration of a prodrug, or by endogenous production), or may be provided to a patient in a form of a pharmaceutical composition. It is not intended that the term “metabolite” of necessity requires production of a compound by a metabolic process in a patient to be treated. Instead, the compound, identified as a “metabolite” may be administered directly to the patient in a pharmaceutical composition rather than in a prodrug form that yields the compound as a metabolite in vivo. The term “metabolite” is employed as a broad term that is not intended to limit compounds to be administered to a patient to compounds produced by a particular method of synthesis (in vivo from a prodrug versus ex vivo).

A small molecule metabolite may be referred to by various names, for example, 2-methylserine may be referred to as 2-amino-3-hydroxy-2-methylpropanoic acid.

In some embodiments, the small molecule metabolite can be an amino acid, peptide, carbohydrate, cofactor and vitamin, xenobiotic, or lipid as provided herein. In further embodiments, one or more small molecule metabolites can include at least one amino acid, peptide, carbohydrate, cofactor and vitamin, xenobiotic, or lipid as provided herein.

Small molecule metabolites that are ideal candidates as both biomarkers and therapeutics are metabolites that are successfully detected in serum at high nanomolar or micromolar levels that have a low molecular weight (<900 daltons) and meet Lipinski's rule of five. All metabolites provided herein meet these criteria.

In some embodiments, a small molecule metabolite can be an amino acid, including but not limited to 2-methylserine, 4-hydroxyglutamate, N-acetyl-aspartyl-glutamate, 2-pyrrolidinone, trans-urocanate, imidazole proprionate, 1-ribosyl-imidazoleacetate, 5-imidazoleacetate, N-acetylhistamine, hydantoin 5-prorionic acid, 5-hydroxylysine, 5-aminovalerate, 2-oxoadipate, xanthurenate, methionine sulfone, homocitrulline, trans-4-hydroxyproline, prolyl-hydroxyproline, or guanidinosuccinate. Derivatives can be synthesized by published methods.

In some embodiments, a small molecule metabolite can be a peptide, including but not limited to gamma-glutamylglutamine, or gamma-glutamylglycine; a carbohydrate, including but not limited to N6-carboxymethyllysine; a cofactor or vitamin, including N1-methyl-2-pyridone-5-carboxamide, N1-methyl-4-pyridone-3-carboxamide; or a xenobiotic, including but not limited to 2,3-dihydroxyisovalerate. Derivatives can be synthesized by published methods.

In some embodiments, a small molecule metabolite can be a monohyroxy fatty acid, including but not limited to 2-hydroxyocatnoate, 2-hydroxydecanoate, 8-hydroxyoctanoate, 2-hydroxymyristate, or 16-hydroxypalmitate. Derivatives can be synthesized by published methods.

In some embodiments, a small molecule metabolite can be a medium chain fatty acid, e.g., a fatty acid including but not limited to one containing a group such as heptanoate (C7:0, e.g., heptanoic acid), caprylate (C8:0, e.g., caprylic acid), pelargonate (C9:0, e.g., pelargonic acid), undecanoate (C11:0, e.g., undecanoic acid), or 10-undecanoate (C11:1n1, e.g., 10-undecanoic acid); a long chain fatty acid, including but not limited to pentadecanoate (C15:0, e.g., pentadecanoic acid), margarate (C17:0, e.g., margaric acid), 10-heptadecanoate (C17:1n7, e.g., 10-heptadecanoic acid), 10-nonadecanoate (19:1n9, e.g., 10-nonadecanoic acid), C20:0 fatty acid, or C20:2 fatty acid; a very long chain fatty acid, including but not limited to C24:0 fatty acid or C24:1 fatty acid; a branched chain fatty acid, including but not limited to 15-methylpalmitate (i17:0, e.g., methylpalmitic acid), 17-methylstearate (i19:0, e.g., 17-methylstearic acid), or 2-hydroxyphytanate (e.g., 2-hydroxyphytanic acid); or a dicarboxylate fatty acid, including but not limited to dodecadienoate (C12:2, e.g., dodecandioic acid) or docosadioate (C22-DC, e.g., docosadioic acid). Derivatives can be synthesized by published methods.

In some embodiments, a small molecule metabolite can be a part or product of fatty acid metabolism, including but not limited to propionylglycine, lignoceroylcarnitine (C24), cerotoylcarnitine (C26), N-palmitoylglycine, cis-4-decenoylcarnitine (C10:1), behenoylcarnitine (C22), pentadecanoylcarnitine (C15), or arachidonoylcholine. Derivatives can be synthesized by published methods.

In some embodiments, a small molecule metabolite can be a phosphatidylcholine, including but not limited to 1-stearoyl-2-arachidonoyl-GPC (18:0/20:4), 1-palmitoyl-2-arachidonoyl-GPC (16:0/20:4n6), PC (18:2/22:4), PC (20:0/14:1), PC (20:0/20:3), or PC (20:0/22:4); a phosphatidylethanolamine, including but not limited 1-palmitoyl-2-arachidonoyl-GPE (16:0/20:4), 1-stearoyl-2-arachidonoyl-GPE (18:0/20:4), or PE (16:0/16:0); a phosphatidylserine, including but not limited to 1-stearoyl-2-oleoyl-GPS (18:0/18:1); a lysophospholipid, including but not limited 1-arachidonoyl-GPC (20:4n6), 1-lignoceroyl-GPC (24:0), or 1-arachidonoyl-GPE (20:4n6). Derivatives can be synthesized by published methods.

In some embodiments, a small molecule metabolite can be a plasmalogen, including but not limited to 1-(1-enyl-palmitoyl)-2-arachidonoyl-GPE (β-16:0/20:4), 1-(1-enyl-palmitoyl)-2-oleoyl-GPC (β-16:0/18:1), 1-(1-enyl-palmitoyl)-2-arachidonoyl-GPC (β-16:0/20:4), or 1-(1-enyl-stearoyl)-2-arachidonoyl-GPE (β-18:0/20:4); or a lysoplasmalogen, including but not limited to 1-(1-enyl-palmitoyl)-GPC (β-16:0). Derivatives can be synthesized by published methods.

In some embodiments, a small molecule metabolite can be a monoacylglycerol (MAG), including but not limited to MAG (12:0), MAG (17:0), MAG (20:0), MAG (20:2), 1-arachidonylglycerol (20:4) or 1-heptadecenoylglycerol (17:1); or a diacylglycerol (DAG), including but not limited to DAG (14:1/18:1), stearoyl-arachidonoyl-glycerol (18:0/20:4) [2], oleoyl-arachidonoyl-glycerol (18:1/20:4) [1], or oleoyl-arachidonoyl-glycerol (18:1/20:4) [2]. Derivatives can be synthesized by published methods.

In some embodiments, a small molecule metabolite can be a sphingomyelin, including but not limited to stearoyl sphingomyelin (d18:1/18:0), behenoyl sphingomyelin (d18:1/22:0), tricosanoyl sphingomyelin (d18:1/23:0), lignoceroyl sphingomyelin (d18:1/24:0), sphingomyelin (d18:2/23:1), sphingomyelin (d18:2/24:2), sphingomyelin (d17:1/14:0, d16:1/15:0), sphingomyelin (d17:1/16:0, d18:1/15:0, d16:1/17:0), sphingomyelin (d17:2/16:0, d18:2/15:0), sphingomyelin (d18:1/17:0, d17:1/18:0, d19:1/16:0), sphingomyelin (d18:1/19:0, d19:1/18:0), sphingomyelin (d18:1/21:0, d17:1/22:0, d16:1/23:0), sphingomyelin (d18:2/21:0, d16:2/23:0), or sphingomyelin (d18:2/23:0, d18:1/23:1, d17:1/24:1). Derivatives can be synthesized by published methods.

In some embodiments, a small molecule metabolite can be a ceramide, including but not limited to CER (14:0), HCER (26:1), or LCER (26:0). Derivatives can be synthesized by published methods.

In some embodiments, a derivative of a small molecule metabolite can be a (3-sulfenyl derivative. It is thought that β-sulfenyl derivatives, such as an acid or ester, can be resistant to β-oxidation in the body. Derivatives can be synthesized by published methods.

In some embodiments, a small molecule metabolite is provided in a bioavailable form. The term “bioavailability” refers to the fraction of an administered dose of unchanged drug that reaches the systemic circulation, one of the principal pharmacokinetic properties of drugs. By definition, when a medication is administered intravenously, its bioavailability is 100%. As employed herein, the term “bioavailable” refers to a form of the small molecule metabolite that is successfully absorbed by the body when using methods of administration other than intravenous, for example, an oral therapeutic). In some embodiments, small molecule metabolite-based compositions may include adaptions that optimize absorption.

A pure or purified small molecule metabolite may exist in various physical states. For example, 4-hydroxyglutamate exists as a white powder that is stable at room temperature; this compound can be purchased in forms suitable for research purposes in small amounts from some commercial suppliers (for example, from Sigma-Aldrich Corp., of St. Louis, MO). Other small molecule metabolites, or stereoisomers, or solvates, or esters, or salts or other derivatives thereof, may exist as oils, solids, crystalline solids, or gases.

A pentadecanoylcarnitine, pentadecanoic acid, or other small molecule metabolites or the pharmaceutically acceptable salts or derivatives thereof, may be provided in a purity (e.g., a percentage of the compound, or its pharmaceutically acceptable salts, in a bulk form) of at least about 10%, at least about 20%, at least about 30%, at least about 40%, at least about 50%, at least about 60%, at least about 70%, at least about 80%, at least about 90%, at least about 95%, at least about 98%, at least about 99%, at least about 99.9%, at least about 99.99%, or substantially pure, wherein substantially pure may include, but not be limited to, a product with impurities at a level such that no physiological effect from the presence of the impurities is detectable. A mixture of small molecule metabolites, such as, for example, an amino acid and/or lipid, or pharmaceutically acceptable salts or derivatives thereof, may be present in a purity of at least about 10%, at least about 20%, at least about 30%, at least about 40%, at least about 50%, at least about 60%, at least about 70%, at least about 80%, at least about 90%, at least about 95%, at least about 98%, at least about 99%, at least about 99.9%, at least about 99.99%, or substantially pure, or ranges including and/or spanning the aforementioned values. The pentadecanoylcarnitine, pentadecanoic acid, or a mixture thereof, or a pharmaceutically acceptable salt thereof, may be free from other small molecule metabolites, singly or taken as a group. In some embodiments, pentadecanoylcarnitine, pentadecanoic acid, or a mixture thereof as provided herein may be substantially free from other species of lipids not included herein.

A pentadecanoylcarnitine, pentadecanoic acid, or small molecule metabolite, such as an amino acid, peptide, carbohydrate, cofactor, vitamins, xenobiotic, or lipid, or a pharmaceutically acceptable salt or derivative thereof, may be from any source. In some embodiments, pentadecanoylcarnitine, pentadecanoic acid, or salts thereof, may be present in natural sources, may be isolated from natural sources, may be semi-synthetic, may be synthetic, or may be a mixture of one or more of these. The pentadecanoylcarnitine, pentadecanoic acid, or salts thereof, may be produced in a laboratory, may be produced in nature, may be produced by enzymatic processes, may be produced by wild microbes, may be produced by genetically modified microbes, may be isolated from animal tissues, may be produced by chemical synthesis, or may be produced by a plurality of these processes.

The pentadecanoylcarnitine, pentadecanoic acid, or small molecule metabolite may be derived from natural sources, e.g., fish oils, or can be synthesized by methods as are known in the art. In some embodiments, the pentadecanoylcarnitine, pentadecanoic acid, or small molecule metabolite may be contaminated with undesired components present in unrefined or unpurified natural products. In such situations, it can be desirable to remove undesired components, or to increase the concentration of desired components using known separation or purification techniques.

In any compound described, all tautomeric forms are also intended to be included. Without limitation, all tautomers of carboxylic groups are intended to be included.

In any compound described herein having one or more double bond(s) generating geometrical isomers that can be defined as E or Z, each double bond may independently be E or Z, or a mixture thereof.

Where compounds disclosed herein have unfilled valencies, then the valencies are to be filled with hydrogens or isotopes thereof, e.g., hydrogen-1 (protium) and hydrogen-2 (deuterium).

The pentadecanoylcarnitine, pentadecanoic acid, or small molecule metabolite, such as an amino acid, peptide, carbohydrate, cofactors and vitamin, xenobiotics, or lipid, as described herein, includes crystalline forms (also known as polymorphs, which include the different crystal packing arrangements of the same elemental composition of a compound), amorphous phases, salts, solvates, and hydrates. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

The compounds described herein can be labeled isotopically. In some circumstances, substitution with isotopes such as deuterium may afford certain therapeutic advantages resulting from greater metabolic stability, such as, for example, increased in vivo half-life or reduced dosage requirements. Isotopic substitution may be beneficial in monitoring subject response to administration of a compound, for example, by providing opportunity for monitoring of the fate of an atom in a compound. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

### Compositions Including Pentadecanoylcarnitine or Pentadecanoic Acid

Formulations including pentadecanoylcarnitine or pentadecanoic acid are provided. It is generally preferred to administer the compounds of the embodiments in oral formulations; however, other routes of administration are also contemplated, such as topical. The formulations are suitable for use as consumer health and wellness products, including over the counter (OTC) products, as well as supplements and foodstuffs.

The compositions of the embodiments may be used in cosmetic, cosmeceutical and general skincare compositions or provided in pharmaceutical compositions.

The compositions of the embodiments can also be employed in the connection with mucous membranes, e.g., the lips and the vaginal mucosa. When applied to the vaginal mucosa, a vaginal applicator can be employed as are commercially available. Suitable applicators can be in a form of a pre-filled syringe, a tube attached to a prefilled squeezable reservoir, a prepackaged wand including a preselected amount of composition, or a universal vaginal applicator including perforations along its length for dispensing the composition through the perforations.

Certain of the compositions can contain further therapeutic agents., e.g., locally acting drugs such as antibacterial drugs, antiprotozoal drugs, antifungal drugs, antiviral drugs, spermicidal agents, prostaglandins, and steroids. Drugs suitable for delivery include bromocriptine, sildenafil, oxytocin, calcitonin, luteinizing hormone-releasing hormone and analogues, insulin, human growth hormone, oxybutynin, and steroids used in hormone replacement therapy or for contraception. Antifungal drugs include clotrimazole, econazole, miconazole, terbinafine, fluconazole, ketoconazole, and amphotericin. Antibiotics include amoxicillin, doxycycline, cephalexin, ciprofloxacin, clindamycin, metronidazole, azithromycin, sulfamethoxazole/trimethoprim, amoxicillin/clavulanate, and levofloxacin. Classes of antibiotics include penicillins, tetracyclines, cephalosporins, quinolones, lincomycins, macrolides, sulfonamides, glycopeptides, aminoglycosides, and carbapenems. Types of hormones include 5-alpha-reductase inhibitors, adrenal cortical steroids, corticotropin, glucocorticoids, mineralocorticoids, adrenal corticosteroid inhibitors, antiandrogens, antidiuretic hormones, antigonadotropic agents, antithyroid agents, inhibitors, calcitonin, estrogen receptor antagonists, gonadotropin-releasing hormone antagonists, growth hormone receptor blockers, growth hormones, insulin-like growth factor, parathyroid hormone and analogs, progesterone receptor modulators, prolactin inhibitors, selective estrogen receptor modulators, sex hormones, androgens and anabolic steroids, contraceptives, estrogens, gonadotropin releasing hormones, gonadotropins, progestins, sex hormone combinations, somatostatin and somatostatin analogs, synthetic ovulation stimulants, and thyroid drugs. Antiviral agents include adamantane antivirals, antiviral boosters, antiviral combinations, antiviral interferons, chemokine receptor antagonist, integrase strand transfer inhibitor, miscellaneous antivirals, neuraminidase inhibitors, NNRTIs, NS5A inhibitors, nucleoside reverse transcriptase inhibitors (NRTIs), protease inhibitors, and purine nucleosides. Drugs for treating skin conditions include acne drugs (isotretinoin), atopic dermatitis drugs (topical steroids), herpes zoster drugs (antivirals such as valacyclovir), hives (antihistamines like loratadine or fexofenadine, omalizumab), sunburn (lidocaine), contact dermatitis (antihistamines, topical steroids), diaper rash (zinc oxide), rosacea (metronidazole, doxycycline, azelaic acid, isotretinoin, beta blockers, estrogen), athlete's foot (antifungals), and basal cell carcinoma (imiquimod, fluorouracil, vismodegib).

The compositions of the embodiments include topical formulations containing at least one excipient. Excipients can include a nonaqueous or aqueous carrier, and one or more agents selected from moisturizing agents, pH adjusting agents, deodorants, fragrances, chelating agents, preservatives, emulsifiers, thickeners, solubilizing agents, penetration enhancers, anti-irritants, colorants, surfactants, beneficial agents, pharmaceutical agents, and other components as known in the art for use in connection with topical formulations for treatment of the skin. The composition can be formulated such that preservatives need not be employed.

To facilitate application, the composition may be provided as an ointment, an oil, a lotion, a paste, a powder, a gel, or a cream. The composition may also include additional ingredients such as a protective agent, an emollient, an astringent, a humectant, a sun screening agent, a sun tanning agent, a UV absorbing agent, an antibiotic agent, an antifungal agent, an antiviral agent, an antiprotozoal agent, an anti-acne agent, an anesthetic agent, a steroidal anti-inflammatory agent, a non-steroidal anti-inflammatory agent, an antipruritic agent, an additional antioxidant agent, a chemotherapeutic agent, an anti-histamine agent, a vitamin or vitamin complex, a hormone, an anti-dandruff agent, an anti-wrinkle agent, an anti-skin atrophy agent, a skin whitening agent, a cleansing agent, and combinations thereof. In a further embodiment, the composition may avoid animal or cellular-based materials to avoid skin irritation. The composition can be applied to the dermis, or to mucous membranes.

Some embodiments include administering the pentadecanoylcarnitine or pentadecanoic acid in topical formulations; however, other routes of administration are also contemplated (e.g., mucosal, subdermal, oral, or the like) in addition to oral administration. Contemplated routes of administration include but are not limited to topical, mucosal, and subcutaneous. Suitable liquid forms include suspensions, emulsions, solutions, and the like. Unit dosage forms can also be provided, e.g., individual packets with a premeasured amount of the formulation, configured for administration to a body part on a predetermined schedule pre-procedure and post-procedure. Unit dosage forms configured for administration twice or three times a day are particularly preferred; however, in certain embodiments it can be desirable to configure the unit dosage form for administration once a day, four times a day, or more.

In some embodiments, the topical and other formulations typically comprise from about 0.001 wt. % or less to about 50 wt. % or more of active ingredient, such as the pentadecanoylcarnitine or pentadecanoic acid or salt thereof, from about 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 wt. % to about 2, 3, 4, 5, 6, 7, 8, 9, 10, 15, 20, 25, 30, 35, 40, or 45 wt. %, or ranges including and/or spanning the aforementioned values.

Compositions and formulations for topical administration can include transdermal patches, ointments, lotions, creams, gels, drops, sprays, liquids, aerosols, and powders. Conventional pharmaceutical carriers, aqueous, powder or oily bases, thickeners and the like may be employed. In certain applications, an ointment, lotion, cream, gel or similar formulation can be provided that can be applied to the skin using the fingers. Such formulations are typically provided in a squeeze tube or bottle or a pot, or in a roll-on, wherein a ball is secured in the top of a container of the formulation, wherein the ball is permitted to roll. By rolling the ball over the skin surface, liquid in the container is transferred to the skin in a controlled manner. An alternative delivery mechanism includes a container with a perforated lid with a mechanism for advancing an extrudable formulation through the lid. In another form, a gel formulation with sufficient structural integrity to maintain its shape is provided, which is advanced up a tube and applied to the skin (e.g., in a stick form). An advantage of the stick form is that only the formulation contacts the skin in the application process, not the fingers or a portion of a container. A liquid or gel can also be placed using an applicator, e.g., a wand, a sponge, a syringe, or other suitable method.

In some embodiments, the pentadecanoylcarnitine or pentadecanoic acid or salt thereof, can be in combination therapy, or in admixture with a suitable carrier, diluent, or excipient, and can contain auxiliary substances such as wetting or emulsifying agents, pH buffering agents, gelling or viscosity enhancing additives, preservatives, scenting agents, colors, and the like, depending upon the route of administration and the preparation desired. Techniques for formulation and administration of the compounds described herein are known to those skilled in the art. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively). Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulations include, without limitation, monoglycerides, diglycerides, sulfatides, lysolecithin, phospholipids, saponin, bile acids, and the like. The presence of such additional components can influence the physical state, solubility, stability, rate of release, rate of clearance, and penetration of active ingredients.

The compositions for topical administration comprise the pentadecanoylcarnitine or pentadecanoic acid or salt thereof and a dermatologically acceptable vehicle. The vehicle may be aqueous or nonaqueous. The dermatologically acceptable vehicle used in the topical composition may be in the form of a lotion, a gel, an ointment, a liquid, a cream, or an emulsion. If the vehicle is an emulsion, the emulsion may have a continuous aqueous phase and a discontinuous nonaqueous or oil phase (oil-in-water emulsion), or a continuous nonaqueous or oil phase and a discontinuous aqueous phase (water-in-oil emulsion). When administered topically in liquid or gel form, a liquid carrier such as water, petroleum, oils of animal or plant origin such as peanut oil, mineral oil, soybean oil, or sesame oil, or synthetic oils can be added to the active ingredient(s). Physiological saline solution, dextrose, or other saccharide solution, or glycols such as ethylene glycol, propylene glycol, or polyethylene glycol are also suitable liquid carriers. The pharmaceutical compositions can also be in the form of oil-in-water emulsions. The oily phase can be a vegetable oil, such as olive or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions can also contain coloring and scenting agents.

In certain embodiments, a silicone elastomer (e.g., dimethicone crosspolymer) is employed to increase delivery and penetration of the pentadecanoylcarnitine or pentadecanoic acid or salt thereof, into the skin. The pharmaceutical excipients used in the topical preparations of the compositions may be selected from the group consisting of solvents, emollients and/or emulsifiers, oil bases, preservatives, antioxidants, tonicity adjusters, penetration enhancers and solubilizers, chelating agents, buffering agents, surfactants, one or more polymers, and combinations thereof.

Suitable solvents for an aqueous or hydrophilic topical formulation include water; ethyl alcohol; isopropyl alcohol; mixtures of water and ethyl and/or isopropyl alcohols; glycerin; ethylene, propylene or butylene glycols; DMSO; and mixtures thereof. Suitable solvents for hydrophobic topical formulations include mineral oils, vegetable oils, and silicone oils. If desired, the compositions as described herein may be dissolved or dispersed in a hydrophobic oil phase, and the oil phase may then be emulsified in an aqueous phase comprising water, alone or in combination with lower alcohols, glycerin, and/or glycols. Anhydrous formulations may also be employed; however, in certain embodiments it may be acceptable to provide water based compositions, or to permit a limited amount of water to be present.

Viscosity of the compositions can be maintained at the selected level using a pharmaceutically acceptable thickening agent. Suitable viscosity enhancers or thickeners which may be used to prepare a viscous gel or cream with an aqueous base include sodium polyacrylate, xanthan gum, polyvinyl pyrrolidone, acrylic acid polymer, carragenans, hydroxyethyl cellulose, hydroxypropyl cellulose, methyl cellulose, ethyl cellulose, propyl cellulose, hydroxypropyl methyl cellulose, polyethoxylated polyacrylamides, polyethoxylated acrylates, and polyethoxylated alkane thiols. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the thickening agent selected. An amount is preferably used that will achieve the selected viscosity. Viscous compositions are normally prepared from solutions by the addition of such thickening agents, or by employing a base that has an acceptable level of viscosity.

Suitable emollients include hydrocarbon oils and waxes such as mineral oil, petrolatum, paraffin, ceresin, ozokerite, microcrystalline wax, polyethylene, squalene, perhydrosqualene, silicone oils, triglyceride esters, acetoglyceride esters, such as acetylated monoglycerides; ethoxylated glycerides, such as ethoxylated glyceryl monostearate; alkyl esters of fatty acids or dicarboxylic acids.

Suitable silicone oils for use as emollients include dimethyl polysiloxanes, methyl(phenyl) polysiloxanes, and water-soluble and alcohol-soluble silicone glycol copolymers. Suitable triglyceride esters for use as emollients include vegetable and animal fats and oils including castor oil, safflower oil, cotton seed oil, corn oil, olive oil, cod liver oil, almond oil, avocado oil, palm oil, sesame oil, and soybean oil.

Suitable esters of carboxylic acids or diacids for use as emollients include methyl, isopropyl, and butyl esters of fatty acids. Specific examples of alkyl esters including hexyl laurate, isohexyl laurate, iso-hexyl palmitate, isopropyl palmitate, decyl oleate, isodecyl oleate, hexadecyl stearate, decyl stearate, isopropyl isostearate, dilauryl lactate, myristyl lactate, and cetyl lactate; and alkenyl esters of fatty acids such as oleyl myristate, oleyl stearate, and oleyl oleate. Specific examples of alkyl esters of diacids include diisopropyl adipate, diisohexyl adipate, bis(hexyldecyl) adipate, and diisopropyl sebacate.

Other suitable classes of emollients or emulsifiers which may be used in the topical formulations include fatty acids, fatty alcohols, fatty alcohol ethers, ethoxylated fatty alcohols, fatty acid esters of ethoxylated fatty alcohols, and waxes.

Specific examples of fatty acids for use as emollients include pelargonic, lauric, myristic, palmitic, stearic, isostearic, hydroxystearic, oleic, linoleic, ricinoleic, arachidic, behenic, and erucic acids. Specific examples of fatty alcohols for use as emollients include lauryl, myristyl, cetyl, hexadecyl, stearyl, isostearyl, hydroxystearyl, oleyl, ricinoleyl, behenyl, and erucyl alcohols, as well as 2-octyl dodecanol.

Specific examples of waxes suitable for use as emollients include lanolin and derivatives thereof including lanolin oil, lanolin wax, lanolin alcohols, lanolin fatty acids, isopropyl lanolate, ethoxylated lanolin, ethoxylated lanolin alcohols, ethoxolated cholesterol, propoxylated lanolin alcohols, acetylated lanolin, acetylated lanolin alcohols, lanolin alcohols linoleate, lanolin alcohols recinoleate, acetate of lanolin alcohols recinoleate, acetate of lanolin alcohols recinoleate, acetate of ethoxylated alcohols esters, hydrogenolysates of lanolin, hydrogenated lanolin, ethoxylated hydrogenated lanolin, ethoxylated sorbitol lanolin, and liquid and semisolid lanolin. Also usable as waxes include hydrocarbon waxes, ester waxes, and amide waxes. Useful waxes include wax esters such as beeswax, spermaceti, myristyl myristate and stearyl stearate; beeswax derivatives, e.g., polyoxyethylene sorbitol beeswax; and vegetable waxes including carnauba and candelilla waxes.

Polyhydric alcohols and polyether derivatives may be used as solvents and/or surfactants in the topical formulations. Suitable polyhydric alcohols and polyethers include propylene glycol, dipropylene glycol, polypropylene glycols 2000 and 4000, poly(oxyethylene-co-oxypropylene) glycols, glycerol, sorbitol, ethoxylated sorbitol, hydroxypropylsorbitol, polyethylene glycols 200-6000, methoxy polyethylene glycols 350, 550, 750, 2000 and 5000, poly[ethylene oxide] homopolymers (100,000-5,000,000), polyalkylene glycols and derivatives, hexylene glycol, 2-methyl-2,4-pentanediol, 1,3-butylene glycol, 1,2,6-hexanetriol, 2-ethyl-1,3-hexanediol, vicinal glycols having 15 to 18 carbon atoms, and polyoxypropylene derivatives of trimethylolpropane.

Polyhydric alcohol esters may be used as emulsifiers or emollients. Suitable polyhydric alcohol esters include ethylene glycol mono- and di-fatty acid esters, diethylene glycol mono- and di-fatty acid esters, polyethylene glycol (200-6000) mono- and di-fatty acid esters, propylene glycol mono- and di-fatty esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

Suitable emulsifiers for use in topical formulations include anionic, cationic, nonionic, and zwitterionic surfactants. Preferred ionic emulsifiers include phospholipids, such as lecithin and derivatives.

The pentadecanoylcarnitine or pentadecanoic acid or salt thereof can be formulated as a liposome. The small molecule metabolite can be a component of the lipid portion of the liposome or can be encapsulated in the aqueous portion of the liposome. The pentadecanoylcarnitine or pentadecanoic acid or salt thereof can also be coformulated with a cyclodextrin. The cyclodextrin can be, for example, hydroxypropyl-β-cyclodextrin or a sulfobutylether cyclodextrin. Lecithin and other phospholipids may be used to prepare liposomes containing active ingredients as described herein. Formation of lipid vesicles occurs when phospholipids such as lecithin are placed in water and consequently form one bilayer or a series of bilayers, each separated by water molecules, once enough energy is supplied. Liposomes can be created by sonicating phospholipids in water. Low shear rates create multilamellar liposomes. Continued high-shear sonication tends to form smaller unilamellar liposomes. Hydrophobic chemicals can be dissolved into the phospholipid bilayer membrane. The lipid bilayers of the liposomes deliver the compositions as described herein.

The topical formulations may contain micelles, or an aggregate of surfactant molecules dispersed in an aqueous solution. Micelles may be prepared by dispersing an oil solvent in an aqueous solution comprising a surfactant, where the surfactant concentration exceeds the critical micelle concentration. The resulting formulation contains micelles, i.e., spherical oil droplets surrounded by a membrane of polar surfactant molecules, dispersed in the aqueous solvent.

Sterols including, for example, cholesterol and cholesterol fatty acid esters; amides such as fatty acid amides, ethoxylated fatty acid amides, and fatty acid alkanolamides may also be used as emollients and/or penetration enhancers.

A pharmaceutically acceptable preservative can be employed to increase the shelf life of the composition. Other suitable preservatives and/or antioxidants for use in topical formulations include benzalkonium chloride, benzyl alcohol, phenol, urea, parabens, butylated hydroxytoluene (BHT), butylated hydroxyanisole (BHA), tocopherol, thimerosal, chlorobutanol, or the like, and mixtures thereof, can be employed. If a preservative, such as an antioxidant, is employed, the concentration is typically from about 0.02% to about 2% based on the total weight of the composition, although larger or smaller amounts can be desirable depending upon the agent selected. Reducing agents, as described herein, can be advantageously used to maintain good shelf life of the formulation. It is generally observed that the anhydrous formulations of the embodiments exhibit satisfactory stability, such that a preservative can be omitted from the formulation.

Suitable chelating agents for use in topical formulations include ethylene diamine tetraacetic acid, alkali metal salts thereof alkaline earth metal salts thereof, ammonium salts thereof, and tetraalkyl ammonium salts thereof.

In some embodiments, the carrier may have a pH of between about 4.0 and 10.0. In some embodiments, the carrier may have a pH of between about 6.8 and about 7.8. The pH may be controlled using buffer solutions or other pH modifying agents. Suitable pH modifying agents include phosphoric acid and/or phosphate salts, citric acid and/or citrate salts, hydroxide salts (i.e., calcium hydroxide, sodium hydroxide, potassium hydroxide) and amines, such as triethanolamine. Suitable buffer solutions include a buffer comprising a solution of monopotassium phosphate and dipotassium phosphate, maintaining a pH of between 5.8 and 8; and a buffer comprising a solution of monosodium phosphate and disodium phosphate, maintaining a pH of between 6 and 7.5. Other buffers include citric acid/sodium citrate, and dibasic sodium phosphate/citric acid. The compositions of the embodiments may be isotonic with the blood or other body fluid of the recipient. The isotonicity of the compositions can be attained using sodium tartrate, propylene glycol or other inorganic or organic solutes. Sodium chloride is particularly preferred. Buffering agents can be employed, such as acetic acid and salts, citric acid and salts, boric acid and salts, and phosphoric acid and salts. It can be desirable to include a reducing agent in the formulation, such as vitamin C, vitamin E, or other reducing agents as are known in the pharmaceutical arts.

Surfactants can also be employed as excipients, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

When the formulations of the embodiments are administered by subcutaneous injection, it is preferably in the form of a pyrogen-free, parenterally acceptable aqueous solution or oleaginous suspension, emulsion or solution. Suspensions can be formulated according to methods well known in the art using suitable dispersing or wetting agents and suspending agents. The preparation of acceptable aqueous or nonaqueous solutions with suitable properties, e.g., pH, isotonicity, stability, and the like, is within the skill in the art. For example, an isotonic vehicle such as 1,3-butanediol, water, isotonic sodium chloride solution, Ringer's solution, dextrose solution, dextrose and sodium chloride solution, lactated Ringer's solution, or other vehicles as are known in the art can be employed, or a fixed oil can be employed conventionally as a solvent or suspending medium, e.g., synthetic mono or diglycerides, fatty acids, or the like. The formulations can also contain stabilizers, preservatives, buffers, antioxidants, or other additives known to those of skill in the art.

In certain embodiments, it can be advantageous to include additional agents having pharmacological activity. Anti-infective agents include, but are not limited to, anthelmintic (mebendazole), antibiotics including aminoglycosides (gentamicin, neomycin, tobramycin), antifungal antibiotics (amphotericin b, fluconazole, griseofulvin, itraconazole, ketoconazole, nystatin, micatin, tolnaftate), cephalosporins (cefaclor, cefazolin, cefotaxime, ceftazidime, ceftriaxone, cefuroxime, cephalexin), beta-lactam antibiotics (cefotetan, meropenem), chloramphenicol, macrolides (azithromycin, clarithromycin, erythromycin), penicillins (penicillin G sodium salt, amoxicillin, ampicillin, dicloxacillin, nafcillin, piperacillin, ticarcillin), tetracyclines (doxycycline, minocycline, tetracycline), bacitracin, clindamycin, colistimethate sodium, polymyxin b sulfate, vancomycin, antivirals including acyclovir, amantadine, didanosine, efavirenz, foscarnet, ganciclovir, indinavir, lamivudine, nelfinavir, ritonavir, saquinavir, stavudine, valacyclovir, valganciclovir, zidovudine, quinolones (ciprofloxacin, levofloxacin), sulfonamides (sulfadiazine, sulfisoxazole), sulfones (dapsone), furazolidone, metronidazole, pentamidine, sulfanilamidum crystallinum, gatifloxacin, and sulfamethoxazole/trimethoprim. Anesthetics can include, but are not limited to, ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocaine, and phenazopyridine. Anti-inflammatory agents include but are not limited to, nonsteroidal anti-inflammatory drugs (NSAIDs) such as aspirin, celecoxib, choline magnesium trisalicylate, diclofenac potassium, diclofenac sodium, diflunisal, etodolac, fenoprofen, flurbiprofen, ibuprofen, indomethacin, ketoprofen, ketorolac, melenamic acid, nabumetone, naproxen, naproxen sodium, oxaprozin, piroxicam, rofecoxib, salsalate, sulindac, and tolmetin; and corticosteroids such as cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinonide, betamethasone dipropionate, betamethasone valerate, desonide, desoximetasone, fluocinolone, triamcinolone, clobetasol propionate, and dexamethasone.

In certain embodiments, the addition of emollients, emulsion stabilizers, moisturizers, excipients, and other compounds may be modified to enhance the sensory properties of the topical compositions, including but not limited to: skin feel (silkiness, lightness, creaminess, etc.), absorbency (required time at which product loses wet feel and is no longer perceived on skin), consistency, firmness, spreadability (e.g. viscosity, flow onset, shear rates), stickiness, integrity of shape, glossiness, hydrophilicity or hydrophobicity, and others.

In certain embodiments systemic administration of the pentadecanoylcarnitine or pentadecanoic acid can be desirable. In such embodiments, the pentadecanoylcarnitine or pentadecanoic acid or salt thereof are formulated into a composition suitable for oral administration, but other routes of administration are also contemplated.

The compositions described herein can be administered by themselves to a subject, or in compositions where they are mixed with other active agents, as in combination therapy, or with carriers, diluents, excipients or combinations thereof. Formulation is dependent upon the route of administration chosen. Techniques for formulation and administration of the compounds described herein are known to those skilled in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

The compositions disclosed herein may be manufactured into administrable forms by a process that is itself known, e.g., by means of conventional mixing, dissolving, granulating, dragee-making, levigating, emulsifying, encapsulating, entrapping, tableting, or extracting processes. Many of the compounds used in the pharmaceutical combinations disclosed herein may be provided as salts with pharmaceutically acceptable counterions.

Multiple techniques of administering pentadecanoylcarnitine or pentadecanoic acid or salt thereof exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections. Contemplated herein is any combination of the forgoing, or other methods as would be known to one of ordinary skill in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

In practice, the pentadecanoylcarnitine or pentadecanoic acid or salt thereof may be combined as the active ingredient in intimate admixture with a pharmaceutical carrier according to conventional pharmaceutical compounding techniques. The pentadecanoylcarnitine or pentadecanoic acid or salt thereof can be added directly to, e.g., a gelatin capsule or a softgel capsule for consumption by the patient. In other embodiments, carriers can be employed. The carrier can take a wide variety of forms depending on the form of preparation desired for administration. Thus, the compositions provided herein can be presented as discrete units suitable for oral administration such as capsules, cachets or tablets each containing a predetermined amount of the active ingredient. Further, the compositions can be presented as an oil, a powder, as granules, as a solution, as a suspension in an aqueous liquid, as a non-aqueous liquid, as an oil-in-water emulsion, or as a water-in-oil liquid emulsion, similar to the topical formulations described elsewhere herein, but using components suitable for human consumption. In addition to the common dosage forms set out above, the compositions provided herein can also be administered by controlled release and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredients with liquid carriers or finely divided solid carriers or both. The product can then be conveniently shaped into the desired presentation.

A formulation may also be administered in a local rather than systemic manner, for example, via injection of the composition directly into a target area, e.g., in a depot or sustained release formulation. Furthermore, a targeted drug delivery system for the composition may be used, for example, in a liposome coated with a tissue specific antibody.

The compositions may contain the pentadecanoylcarnitine or pentadecanoic acid or salt thereof in an amount effective for the desired therapeutic effect. In some embodiments, the compositions are in a unit dosage form and comprise from about 0.1 mg or less to about 5000 mg or more of pentadecanoylcarnitine or pentadecanoic acid or salt thereof per unit dosage form. In further embodiments, the compositions comprise from about 1 to about 500 mg per unit dosage form or from about 500 to 5000 mg per unit dosage form of pentadecanoylcarnitine or pentadecanoic acid or salt thereof. Such dosage forms may be solid, semisolid, liquid, an emulsion, or adapted for delivery via aerosol or the like for inhalation administration.

The carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, lower alcohols, and water. Examples of gaseous carriers include carbon dioxide and nitrogen.

The compositions provided herein can be prepared as solutions or suspensions of the pentadecanoylcarnitine or pentadecanoic acid or salt thereof in water or nonaqueous liquids. A suitable surfactant can be included such as, for example, hydroxypropylcellulose. Dispersions can also be prepared in glycerol, liquid polyethylene glycols, and mixtures thereof in oils. Further, a preservative can be included to, for example, prevent the detrimental growth of microorganisms.

Compositions provided herein suitable for injectable use include sterile aqueous solutions or dispersions. Furthermore, the compositions can be in the form of sterile powders for the extemporaneous preparation of such sterile injectable solutions or dispersions. The compositions may be stable under the conditions of manufacture and storage; thus, may be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

In addition to the aforementioned carrier ingredients, the formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood or other bodily fluids of the intended recipient. Compositions containing a compound provided herein, or pharmaceutically acceptable salt or derivative thereof, can also be prepared in powder or liquid concentrate form for dilution.

Contemplated herein are compositions including the pentadecanoylcarnitine or pentadecanoic acid or salt thereof as described herein in combination with at least one additional active agent. The pentadecanoylcarnitine or pentadecanoic acid or salt thereof and the at least one additional active agent(s) may be present in a single formulation or in multiple formulations provided together, or may be unformulated. In some embodiments, the pentadecanoylcarnitine or pentadecanoic acid or salt thereof can be administered with one or more additional agents together in a single composition. For example, the pentadecanoylcarnitine or pentadecanoic acid or salt thereof can be administered in one composition, and at least one of the additional agents can be administered in a second composition. In a further embodiment, the pentadecanoylcarnitine or pentadecanoic acid or salt thereof and the at least one additional active agent(s) are co-packaged in a kit. For example, a drug manufacturer, a drug reseller, a physician, a compounding shop, or a pharmacist can provide a kit comprising the pentadecanoylcarnitine or pentadecanoic acid or salt thereof in combination with another product or component for delivery to a patient. Such additional components can include anti-infective agents, anti-inflammatory agents, anesthetics, or the like.

Some embodiments described herein relate to oral compositions of pentadecanoylcarnitine or pentadecanoic acid or salt thereof which can include a therapeutically effective amount of the pentadecanoylcarnitine or pentadecanoic acid or salt thereof described herein and a pharmaceutically acceptable carrier, diluent, excipient or combination thereof. The compositions can include the pentadecanoylcarnitine or pentadecanoic acid or salt thereof in an amount for example, ≥1%, ≥2%, ≥3%, ≥4%, ≥5%, ≥6%, ≥7%, ≥8%, ≥9%, ≥10%, ≥20%, ≥30%, ≥40%, ≥50%, ≥60%, ≥70%, ≥80%, ≥90%, ≥95%, ≥98% of the composition or ranges including and/or spanning the aforementioned values. In some embodiments, the pharmaceutical composition can include pentadecanoylcarnitine or pentadecanoic acid or salt thereof and one or more other small molecule metabolites, such as one or more of an amino acid, peptide, carbohydrate, cofactor, vitamins, xenobiotic, and/or lipid described herein, or salts or derivatives thereof in, for example, ≥1%, ≥2%, ≥3%, ≥4%, ≥5%, ≥6%, ≥7%, ≥8%, ≥9%, ≥10%, ≥20%, ≥30%, ≥40%, ≥50%, ≥60%, ≥70%, ≥80%, ≥90%, ≥95%, ≥98% of the composition, or ranges including and/or spanning the aforementioned values.

### Foodstuffs

Foodstuffs and other comestibles including pentadecanoylcarnitine or pentadecanoic acid, or a salt thereof, are provided, wherein an amount of the pentadecanoylcarnitine or pentadecanoic acid, or a salt thereof in the foodstuff has been fortified (e.g., enriched or concentrated). Pentadecanoylcarnitine or pentadecanoic acid, or a salt thereof may be added to foodstuffs for consumption by a subject. The pentadecanoylcarnitine or pentadecanoic acid, or a salt thereof may be integrated into one or more ingredients of a foodstuff. The pentadecanoylcarnitine or pentadecanoic acid, or a salt thereof may be prepared as an ingredient, or may be unprepared. The pentadecanoylcarnitine or pentadecanoic acid, or a salt thereof, or preparation including the pentadecanoylcarnitine or pentadecanoic acid, or a salt thereof, may be added prior to preparation, during preparation, or following preparation. Preparation may without limitation include cooking, mixing, flavoring, seasoning, blending, boiling, frying, baking, or other processes known in the art. Fortification is preferably at a level so as to provide a therapeutic daily dosage of pentadecanoylcarnitine or pentadecanoic acid, or a salt thereof as described elsewhere herein, or to achieve a body concentration of pentadecanoylcarnitine as described herein; however, beneficial effects may also be obtained at amounts below such dosages. Pentadecanoylcarnitine or pentadecanoic acid, or a salt thereof can be administered as a dietary supplement in a unit dosage form (a tablet, a capsule, an encapsulated pill, or gelcap pill), or in a dispensable form of an oral or injectable liquid suspension or solution, a spray, an aerosol, powder, or granules), or as a dietary supplement, additive, ingredient, or fortifier added to a comestible substance (food or beverage). In certain embodiments, pentadecanoylcarnitine or pentadecanoic acid, or a salt thereof as a dietary supplement or food/beverage ingredient or additive can be utilized to promote or support health, e.g., to promote or support metabolic health, to promote or support heart health, to promote or support liver health, to promote or support red blood cell, to promote or support immune health; and/or slow an aging rate. Accordingly, pentadecanoylcarnitine or pentadecanoic acid, or a salt thereof is suitable for administration in those forms and by those methods known in the pharmaceutical and nutraceutical arts, including but not limited to a dietary supplement, a medical food, a food additive, a food ingredient, a food fortifier, a beverage additive, a beverage ingredient, a beverage fortifier, a fortified food, a fortified beverage, an additized food, an additized beverage, as well as pharmaceutical drug in any form, including as a tablet, encapsulated pill, gelcap pill, liquid suspension, liquid solution, spray, or powder.

Pentadecanoylcarnitine or pentadecanoic acid or salt thereof as provided herein may be present as a constituency in foodstuffs by operation of processes known in nature, for example, by altering the metabolic processes of a plant, animal, bacteria, or fungus. Genetic alteration of a plant, animal, bacteria, or fungus to increase the concentration of pentadecanoylcarnitine or pentadecanoic acid or salt thereof, is contemplated. By way of example, the pentadecanoylcarnitine or pentadecanoic acid or salt thereof can be present in the foodstuff in a concentration of at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, or higher, for example, 1% to 2% or 3% or 4% or 5% or 6% or 7% or 8% or 9% or 10% or 20% or 30% or 40%, 50%, or ranges including and/or spanning the aforementioned values. The pentadecanoylcarnitine or pentadecanoic acid or salt thereof, if naturally present in a foodstuff, can be present in an enriched amount above that which is naturally occurring for the foodstuff, e.g., a concentration of 10% or more above the average or highest naturally occurring observed concentration, e.g., 20% or 30% or 40% or 50% or 100% or 200% or 300% or 400% or 1000% or 2000% or 5000% or more above the average or highest naturally occurring observed concentration.

### Indications

Provided are compositions and methods for treatment, management, amelioration, or prophylaxis of a condition selected from the group consisting of aggression, allergies, allergic rhinitis, Alzheimer's disease, anxiety, anxiety disorders, amyotrophic lateral sclerosis, arthritis, asthma, atherosclerosis, attention-deficit hyperactivity disorder, bipoloar disorder, brain damage, cancer, cardiovascular disease, cholestatic pruritis, depression, chronic obstructive pulmonary disease (COPD), cocaine abuse, cough, dermatitis, depression, drug-seeking behavior, gastrointestinal disorders, facial erythema associated with rosacea, glaucoma, hepatic diseases, hyperactive bladder, hypersensitivity disorders, hypertension, impulsivity, inflammation, mental disorders, mental conditions, metabolic disorders, migraines, nasal congestion, sinus congestion, nausea, neuropathic pain with multiple sclerosis, symptoms of multiple sclerosis, neurological disorders, neuropsychiatric disorders, obesity, obsessive-compulsive disorders, opioid-induced respiratory depression, osteoarthritis, pain, Parkinson disease, pathological gambling, peptic ulcers, schizophrenia, sleep disorders, spinal cord injury, tardive dyskinesia, tics with Tourette's syndrome, behavioral problems with Tourette's syndrome.

Provided are compositions and methods for supporting appetite, cardiovascular health, hematologic health, memory, metabolic health, mood, prolonged REM sleep, renal health, sexuality, sociability, metabolic health, hematological health, renal health, and weight loss.

Provided are compositions and methods for achieving a body concentration of pentadecanoylcarnitine of from 1 μM to 20 μM is achieved in a subject.

In some embodiments, levels of serum, plasma, or erythrocyte membrane pentadecanoylcarnitine may increase following administration pentadecanoylcarnitine or pentadecanoic acid or salt thereof.

In some embodiments, the compositions and methods provided herein modulate a marker of aggression, allergies, allergic rhinitis, Alzheimer's disease, anxiety, anxiety disorders, amyotrophic lateral sclerosis, arthritis, asthma, atherosclerosis, attention-deficit hyperactivity disorder, bipoloar disorder, brain damage, cancer, cardiovascular disease, cholestatic pruritis, depression, chronic obstructive pulmonary disease (COPD), cocaine abuse, cough, dermatitis, depression, drug-seeking behavior, gastrointestinal disorders, facial erythema associated with rosacea, glaucoma, hepatic diseases, hyperactive bladder, hypersensitivity disorders, hypertension, impulsivity, inflammation, mental disorders, mental conditions, metabolic disorders, migraines, nasal congestion, sinus congestion, nausea, neuropathic pain with multiple sclerosis, symptoms of multiple sclerosis, neurological disorders, neuropsychiatric disorders, obesity, obsessive-compulsive disorders, opioid-induced respiratory depression, osteoarthritis, pain, Parkinson disease, pathological gambling, peptic ulcers, schizophrenia, sleep disorders, spinal cord injury, tardive dyskinesia, tics with Tourette's syndrome, or behavioral problems with Tourette's syndrome, or a marker associated with appetite, cardiovascular health, hematologic health, memory, metabolic health, mood, prolonged REM sleep, renal health, sexuality, sociability, metabolic health, hematological health, renal health, or weight loss. In certain embodiments, the marker is serum, plasma, or red blood cell membrane pentadecanoylcarnitine percentage; serum, plasma, or red blood cell membrane concentration of pentadecanoylcarnitine contained herein; or serum plasma, or red blood cell membrane total pentadecanoylcarnitine. In some embodiments, the pentadecanoylcarnitine is measured as a constituent of glycolipids. In further embodiments, the pentadecanoylcarnitine is measured as a constituent of phospholipids. In still further embodiments, the marker is serum or red blood cell membrane pentadecanoylcarnitine percentage, serum concentration of pentadecanoylcarnitine, or serum total pentadecanoylcarnitine.

In some embodiments, the methods provided herein include the step of measuring the concentration of a marker. One of skill in the art will be able to perform suitable methods for such measurements, including but not limited to those described herein.

Provided herein are methods for treating including the step of administering a dose of pentadecanoylcarnitine or pentadecanoic acid or salt thereof, at a predetermined interval, or at an interval left to the discretion of the subject.

In some embodiments, the compounds and methods provided herein may provide a threshold serum, plasma, or red blood cell membrane percentage of pentadecanoylcarnitine relative to all serum, plasma, or red blood cell membrane small molecule metabolites. For example, the threshold value may be a value of about 0.05% or lower to 90% or higher, e.g., a value of at least about 0.05%, at least about 0.1%, at least about 0.2%, at least about 0.3%, at least about 0.4%, at least about 0.5%, at least about 0.6%, at least about 0.7%, at least about 0.8%, at least about 0.9%, at least about 1.0%, at least about 1.1%, at least about 1.2%, at least about 1.3%, at least about 1.4%, at least about 1.5%, at least about 1.6%, at least about 1.7%, at least about 1.8%, at least about 1.9%, at least about 2.1%, at least about 2.2%, at least about 2.3%, at least about 2.4%, at least about 2.5%, at least about 2.6%, at least about 2.7%, at least about 2.8%, at least about 2.9%, at least about 3.0%, at least about 3.5%, at least about 4.0%, at least about 4.5%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, more than 90%, or ranges including and/or spanning the aforementioned values.

In some embodiments, the compositions and methods provided herein may provide an increase above a baseline value (e.g., pretreatment value in a patient being treated, or general value observed in a particular patient population) in a serum or plasma concentration of pentadecanoylcarnitine, or red blood cell membrane concentration of pentadecanoylcarnitine. For example, a serum or plasma pentadecanoylcarnitine or red blood cell membrane concentration of pentadecanoylcarnitine may be increased by at least about 1 μg/ml, at least about 2 μg/ml, at least about 3 μg/ml, at least about 4 μg/ml, at least about 5 μg/ml, at least about 6 μg/ml, at least about 7 μg/ml, at least about 8 μg/ml, at least about 9 μg/ml, at least about 10 μg/ml, at least about 15 μg/ml, at least about 20 μg/ml, at least about 25 μg/ml, at least about 30 μg/ml, at least about 35 μg/ml, at least about 40 μg/ml, at least about 45 μg/ml, at least about 50 μg/ml, more than 50 μg/ml, or ranges including and/or spanning the aforementioned values. In some embodiments, the serum concentration of pentadecanoylcarnitine, or red blood cell membrane concentration of pentadecanoylcarnitine may increase above a baseline value (e.g., pretreatment value in a patient being treated, or general value observed in a particular patient population) by at least about 0.01×10⁻⁴ M, at least about 0.05×10⁻⁴ M, at least about 0.1×10⁻⁴ M, at least about 0.2×10⁻⁴ M, at least about 0.3×10⁻⁴ M, at least about 0.4×10⁻⁴ M, at least about 0.5×10⁻⁴ M, at least about 0.6×10⁻⁴ M, at least about 0.7×10⁻⁴ M, at least about 0.8×10⁻⁴ M, at least about 0.9×10⁻⁴ M, at least about 1×10⁻⁴ M, at least about 2×10⁻⁴ M, at least about 3×10⁻⁴ M, or ranges including and/or spanning the aforementioned values.

In some embodiments, the compounds and methods provided herein may provide an increase in serum or plasma total pentadecanoylcarnitine and pentadecanoic acid, or red blood cell membrane total pentadecanoylcarnitine and pentadecanoic acid. For example, serum total pentadecanoylcarnitine and pentadecanoic acid, or red blood cell membrane total pentadecanoylcarnitine and pentadecanoic acid, may be increased above a baseline value (e.g., pretreatment value in a patient being treated, or general value observed in a particular patient population) by at least about 5 μg/ml, at least about 6 μg/ml, at least about 7 μg/ml, at least about 8 μg/ml, at least about 9 μg/ml, at least about 10 μg/ml, at least about 15 μg/ml, at least about 20 μg/ml, at least about 25 μg/ml, at least about 30 μg/ml, at least about 35 μg/ml, at least about 40 μg/ml, at least about 45 μg/ml, at least about 50 μg/ml, at least about 60 μg/ml, at least about 70 μg/ml, at least about 80 μg/ml, at least about 90 μg/ml, at least about 100 μg/ml, at least about 150 μg/ml, at least about 200 μg/ml, at least about 250 μg/ml, at least about 300 μg/ml, at least about 350 μg/ml, at least about 400 μg/ml, at least about 450 μg/ml, at least about 500 μg/ml, or more than 500 μg/ml.

In some embodiments, the compounds and methods provided herein may provide an increase above a baseline value (e.g., pretreatment value in a patient being treated, or general value observed in a particular patient population) in a serum, plasma, or red blood cell membrane pentadecanoylcarnitine relative to all serum or red blood cell membrane small molecule metabolites, respectively. For example, a serum, plasma, or red blood cell membrane pentadecanoylcarnitine may be increased above a baseline value (e.g., pretreatment value in a patient being treated, or general value observed in a particular patient population) by at least about 0.01%, at least about 0.05%, at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 1.1%, at least about 1.2%, at least about 1.3%, at least about 1.4%, at least about 1.5%, at least about 1.6%, at least about 1.7%, at least about 1.8%, at least about 1.9%, at least about 2%, at least about 2.1%, at least about 2.2%, at least about 2.3%, at least about 2.4%, at least about 2.5%, at least about 2.6%, at least about 2.7%, at least about 2.8%, at least about 2.9%, at least about 3%, at least about 3.5%, at least about 4%, at least about 4.5%, at least about 5%, more than 5%, or ranges including and/or spanning the aforementioned values.

In some embodiments, pentadecanoylcarnitine or pentadecanoic acid or salt thereof is administered to maintain serum or plasma total percent of pentadecanoylcarnitine above a predetermined threshold value. In further variations, the pentadecanoylcarnitine or pentadecanoic acid or salt thereof is administered to maintain serum phospholipid percent of the pentadecanoylcarnitine above about 0.1%, about 0.2%, about 0.3%, about 0.4%, about 0.5%, about 0.6%, about 0.7%, about 0.8%, about 0.9%, about 1%, about 1.2%, about 1.4%, about 1.6%, about 1.8%, about 2%, about 2.2%, about 2.4%, about 2.6%, or ranges including and/or spanning the aforementioned values.

In some embodiments, the compounds and methods provided herein may provide a threshold serum, plasma, or red blood cell membrane percentage of pentadecanoylcarnitine relative to all serum or red blood cell membrane small molecule metabolites, respectively. For example, the threshold value may be a value of about 0.05% or lower to 90% or higher, e.g., a value of at least about 0.05%, at least about 0.1%, at least about 0.2%, at least about 0.3%, at least about 0.4%, at least about 0.5%, at least about 0.6%, at least about 0.7%, at least about 0.8%, at least about 0.9%, at least about 1.0%, at least about 1.1%, at least about 1.2%, at least about 1.3%, at least about 1.4%, at least about 1.5%, at least about 1.6%, at least about 1.7%, at least about 1.8%, at least about 1.9%, at least about 2.1%, at least about 2.2%, at least about 2.3%, at least about 2.4%, at least about 2.5%, at least about 2.6%, at least about 2.7%, at least about 2.8%, at least about 2.9%, at least about 3.0%, at least about 3.5%, at least about 4.0%, at least about 4.5%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, more than 90%, or ranges including and/or spanning the aforementioned values.

In some embodiments, the compounds and methods provided herein may provide an increase above a baseline value (e.g., pretreatment value in a patient being treated, or general value observed in a particular patient population) in a serum or plasma concentration of pentadecanoylcarnitine, or red blood cell membrane concentration of a pentadecanoylcarnitine. For example, a serum pentadecanoylcarnitine or red blood cell membrane concentration of pentadecanoylcarnitine may be increased by at least about 0.01 μg/ml, at least about 0.05 μg/ml, at least about 0.1 μg/ml, at least about 0.4 μg/ml, 1 μg/ml, at least about 2 μg/ml, at least about 3 g/ml, at least about 4 μg/ml, at least about 5 μg/ml, at least about 6 μg/ml, at least about 7 μg/ml, at least about 8 μg/ml, at least about 9 μg/ml, at least about 10 μg/ml, at least about 15 μg/ml, at least about 20 μg/ml, at least about 25 μg/ml, at least about 30 μg/ml, at least about 35 μg/ml, at least about 40 μg/ml, at least about 45 μg/ml, at least about 50 μg/ml, more than 50 μg/ml, or ranges including and/or spanning the aforementioned values. In some embodiments, the serum concentration of pentadecanoylcarnitine, or red blood cell membrane concentration of pentadecanoylcarnitine may increase above a baseline value (e.g., pretreatment value in a patient being treated, or general value observed in a particular patient population) by at least about 0.001×10⁻⁴ M, at least about 0.005×10⁻⁴ M, at least about 0.05×10⁻⁴ M, at least about 0.01×10⁻⁴ M, at least about 0.05×10⁻⁴ M, at least about 0.1×10⁻⁴ M, at least about 0.2×10⁻⁴ M, at least about 0.3×10⁻⁴ M, at least about 0.4×10⁻⁴ M, at least about 0.5×10⁻⁴ M, at least about 0.6×10⁻⁴ M, at least about 0.7×10⁻⁴ M, at least about 0.8×10⁻⁴ M, at least about 0.9×10⁻⁴ M, at least about 1×10⁻⁴ M, at least about 2×10⁻⁴ M, at least about 3×10⁻⁴ M, or ranges including and/or spanning the aforementioned values.

In some embodiments, the compounds and methods provided herein may provide an increase in serum or plasma total pentadecanoylcarnitine and pentadecanoic acid or salt thereof, or red blood cell membrane total pentadecanoylcarnitine and pentadecanoic acid or salt thereof. For example, serum total pentadecanoylcarnitine and pentadecanoic acid or salt thereof, or red blood cell membrane total pentadecanoylcarnitine and pentadecanoic acid or salt thereof, may be increased above a baseline value (e.g., pretreatment value in a patient being treated, or general value observed in a particular patient population) by at least about 0.05 μg/ml, at least about 0.1 μg/ml, at least about 0.5 μg/ml, at least about 1 μg/ml, at least about 5 μg/ml, at least about 6 μg/ml, at least about 7 μg/ml, at least about 8 μg/ml, at least about 9 μg/ml, at least about g/ml, at least about 15 μg/ml, at least about 20 μg/ml, at least about 25 μg/ml, at least about g/ml, at least about 35 μg/ml, at least about 40 μg/ml, at least about 45 μg/ml, at least about 50 μg/ml, at least about 60 μg/ml, at least about 70 μg/ml, at least about 80 μg/ml, at least about 90 μg/ml, at least about 100 μg/ml, at least about 150 μg/ml, at least about 200 μg/ml, at least about 250 μg/ml, at least about 300 μg/ml, at least about 350 μg/ml, at least about 400 μg/ml, at least about 450 μg/ml, at least about 500 μg/ml, more than 500 μg/ml, or ranges including and/or spanning the aforementioned values.

### Combination Therapies

In some embodiments, the pentadecanoylcarnitine or pentadecanoic acid or salt thereof may be used in combination with one or more additional active agents. Examples of additional active agents that can be used in combination include a small molecule metabolite, or a salt or derivative thereof, or a composition that includes a compound of a small molecule metabolite, or a salt or derivative thereof, include, but are not limited to, agents currently used for treating conditions provided herein, and as otherwise known to medical science.

In some embodiments, pentadecanoylcarnitine or pentadecanoic acid or salt thereof, can be used with one, two, three or more additional active agents described herein. Such agents include, but are not limited to, a small molecule metabolite, or a salt or derivative thereof.

In some embodiments, pentadecanoylcarnitine or pentadecanoic acid or salt thereof can be used (for example, administered or ingested) in combination with another agent or agents for treatment, prevention, maintenance, or prophylaxis of a condition provided herein.

Additionally, pentadecanoylcarnitine or pentadecanoic acid or salt thereof can be used in combination with one or more agents selected from Altoprev (lovastatin), Crestor (rosuvastatin), Lescol (fluvastatin), Lipitor (atorvastatin), Livalo (pitavastatin), Pravachol (pravastatin), Zocor (simvastatin), an anti-platelet medication, a beta blocker, an ACE inhibitor, a calcium channel blocker, a diuretic, anticoagulants, aspirin, bile acid sequestrants, Ezetimibe, Fibrates, Glycoprotein IIb/IIIa Receptor Inhibitors, Niacin (Nicotinic Acid), Nitrates, Platelet Inhibitors, Thrombolytics, lisinopril oral, atenolol oral, Bystolic oral, Diovan oral, hydrochlorothiazide oral, metoprolol succinate oral, amlodipine oral, Norvasc oral, Toprol XL oral, Benicar oral, metoprolol tartrate oral, losartan oral, lisinopril-hydrochlorothiazide oral, clonidine HCl oral, Diovan HCT oral, Cozaar oral, propranolol oral, spironolactone oral, Azor oral, carvedilol oral, Coreg oral, Benicar HCT oral, Exforge oral, Avapro oral, Lotrel oral, verapamil oral, furosemide oral, Lasix oral, Hyzaar oral, Tekturna oral, enalapril maleate oral, Micardis oral, losartan-hydrochlorothiazide oral, ramipril oral, Lopressor oral, Altace oral, Micardis HCT oral, Avalide oral, diltiazem oral, triamterene-hydrochlorothiazide oral, labetalol oral, terazosin oral, amlodipine-benazepril oral, hydralazine oral, Atacand oral, benazepril oral, Tribenzor oral, triamterene oral, doxazosin oral, nifedipine oral, Ziac oral, Aldactone oral, Maxzide oral, Cartia XT oral, prazosin oral, Cardizem CD oral, Zestril oral, Dyazide oral, bisoprolol fumarate oral, Tenex oral, Tenormin oral, Coreg CR oral, Prinivil oral, valsartan oral, atenolol-chlorthalidone oral, Edarbyclor oral, benazepril-hydrochlorothiazide oral, ferrous sulfate oral, Ferrlecit intravenous, Feraheme intravenous, Feosol oral, Infed injection, Integra oral, Ferrex 150 Forte oral, Tandem Dual Action oral, Ferrex 150 oral, ferrous gluconate oral, Corvite 150 oral, Integra F oral, NovaFerrum oral, Iron (ferrous sulfate) oral, Vitron-C oral, Folic acid, corticosteroids, rituximab, IVIG, prednisone, methylprednisolone oral, Kenalog injection, Medrol (Pak) oral, Medrol oral, dexamethasone oral, Depo-Medrol injection, prednisolone oral, DexPak 13 Day oral, Solu-Medrol intravenous, hydrocortisone oral, Cortef oral, Deltasone oral, triamcinolone acetonide injection, cortisone oral, cholinesterase inhibitors such as Donepezil (Aricept), Rivastigmine (Exelon), and Galantamine (Razadyne), Memantine, Aricept, Namenda, Namenda XR, Razadyne ER, Alpha E, vitamin E, Hydergine, Namzaric, Dopamine Agonists such as pramipexole (Mirapex), ropinirole (Requip), rotigotine (Neupro patch) and apomorphine (Apokyn), Anticholinergics such as benztropine (Cogentin) and trihexyphenidyl, MAO-B Inhibitors such as (Eldepryl, Zelapar) and rasagiline (Azilect), COMT Inhibitors such as Entacapone (Comtan), Carbidopa/Levodopa (Sinemet®), amantadine, Tetrabenazine (Xenazine), haloperidol (Haldol), chlorpromazine, risperidone (Risperdal), quetiapine (Seroquel), olanzapine (Zyprexa), indomethacin, sulindac, etodolac, mefenamic acid, meclofenamic acid, meclofenamate sodium, flufenamic acid, tolmetin, ketorolac, diclofenac, diclofenac sodium, ibuprofen, naproxen, naproxen sodium, fenoprofen, ketoprofen, flurbiprofen, oxaprozin piroxicam, meloxicam, ampiroxicam, droxicam, lornoxicam, cinnoxicam, sudoxicam, and tenoxicam.

Additionally, pentadecanoylcarnitine or pentadecanoic acid or salt thereof can be used in combination with one or more agents selected from iron dextran, iron sumalate, polysaccharide iron, ferrus fumarate, carbonyl iron, ferrous asparto glycinate, heme iron polypeptide can be sometimes indicated, ferrus bisglycinate as can be the administration of other medicaments such as androgen hormones, such as erythropoietin, folic acid, vitamin B12, vitamin C, succinic acid, niacin, pyridoxine, riboflavin, biotin, thiamine, calcium formate, Aminoxin, Anadrol-50, Chromagen Forte, Epoetin alfa, Epogen, Fe C Tab Plus, FeRiva, FeRivaFA, Ferocon, Ferotrin, Ferralet 90, Ferrex 28, Ferrogels Forte, FoliTab 500, Fumatinic, Hematogen Forte, Hemetab, Integra Plus, Irospan 42/6, Lenalidomide, Maxaron Forte, Myferon 150 Forte, MyKidz Iron, NovaFerrum, Oxymetholone, Procrit, Proferrin-Forte, Pyridoxine, Repliva 21/7, Revlimid, and Tricon.

### Dosing

As will be readily apparent to one skilled in the art, the useful in vivo dosage to be administered and the particular mode of administration will vary depending upon the age, weight, the severity of the condition, and mammalian species treated, the particular forms of the compounds employed, and the specific use for which these compounds are employed. The determination of effective dosage levels (the dosage levels necessary to achieve the desired result) can be accomplished by one skilled in the art using routine methods, for example, in vivo studies. Reference may be made to, for example, “Estimating the Maximum Safe Starting Dose in Initial Clinical Trials for Therapeutics in Adult Healthy Volunteers,” U.S. Food and Drug Administration, July 2005.

In some embodiments, a method provided herein may comprise administering a therapeutically effective amount of a composition provided herein. In some embodiments, a therapeutically effective amount may be determined by reference to the modulation of a marker of a condition provided herein. In some embodiments, a therapeutically effective amount may be determined by reference to the modulation of a symptom of a condition provided herein. In still other embodiments, reference may be made to established guidelines for the conditions described herein, including, but not limited to, guidelines for the treatment of a condition provided herein including inflammation.

The dosage may vary broadly, depending upon the desired effects and the therapeutic indication, such as marker values. Alternatively, dosages may be based and calculated upon the surface area or weight of the patient, as understood by those of skill in the art. The exact dosage will be determined on a case-by-case basis, or, in some cases, will be left to the informed discretion of the subject. The daily dosage regimen for an adult human patient may be, for example, an oral dose of pentadecanoylcarnitine or pentadecanoic acid, or a salt thereof, or a mixture of pentadecanoylcarnitine and pentadecanoic acid, or salts thereof, from about 0.01 mg to about 10000 mg, from about 1 mg to about 5000 mg, from about 5 mg to about 2000 mg, from about 10 mg to about 1000 mg, from about 50 mg to about 500 mg, or from about 100 mg to about 200 mg. A single dose, e.g., a minimum dose, may include pentadecanoylcarnitine or pentadecanoic acid, or a salt thereof, in about 0.01 mg, about 0.1 mg, about 1 mg, about 5 mg, about 10 mg, about 20 mg, about 50 mg, about 100 mg, about 200 mg, about 300 mg, about 400 mg, about 500 mg, about 600 mg, about 800 mg, about 900 mg, about 1000 mg, about 2000 mg, about 5000 mg, or more, or ranges including and/or spanning the aforementioned values. The dosage may be adjusted according to the body mass of the subject, for example, the dosage, e.g., a minimum dosage, may be about 0.001 mg/kg, about 0.01 mg/kg, about 0.1 mg/kg, 0.3 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, or higher or ranges including and/or spanning the aforementioned values. For example, the dosage may be from about 0.001 mg/kg to about 30 mg/kg, from about 0.01 mg/kg to about 10 mg/kg, from about 0.1 mg/kg to about 5 mg/kg, from about 0.3 mg/kg to about 3 mg/kg, or from about 1 mg/kg to about 3 mg/kg. The dosage may be a single one or a series of two or more given in the course of one or more days, as is appropriate for the individual subject. In some embodiments, the pentadecanoylcarnitine or pentadecanoic acid, or a salt thereof will be administered for a period of continuous therapy, for example for about a week or more (e.g., one week, two weeks, three weeks, four weeks, five weeks, six weeks, seven weeks, eight weeks, or more), for several weeks, for about a month or more (e.g., one month, two months, three months, four months, five months, six months, seven months, eight months, nine months, ten months, eleven months, twelve months, or more), for about a year or more, or for a plurality of years. In some embodiments, pentadecanoylcarnitine or pentadecanoic acid, or a salt thereof, can be administered or ingested one time per day, two times per day, three times per day, or more.

As will be understood by those of skill in the art, in certain situations it may be necessary to administer the compounds disclosed herein in amounts that exceed the above-stated, preferred dosage range in order to effectively treat a subject.

Unit dosage forms can also be provided, e.g., individual packages with a premeasured amount of the composition, configured for administration on a predetermined schedule. Unit dosage forms configured for administration one to three times a day are preferred; however, in certain embodiments it may be desirable to configure the unit dosage form for administration more than three times a day, or less than one time per day.

Dosage amount and interval may be adjusted to the individual subject to provide plasma levels of the active moiety which are sufficient to maintain predetermined parameters, indicators, or marker values, or minimal effective concentration (MEC). Dosages necessary to achieve the desired result will depend on individual characteristics and route of administration. However, assays, for example, HPLC assays or bioassays, may be used to determine serum concentrations.

In some embodiments, the compounds and methods provided herein may be used in conjunction with devices and methods of using devices, for example, as provided in U.S. Pat. Nos. 7,651,845; 8,251,904; 8,251,904; 4,985,015; 8,827,957; 4,252,159; 5,318,521; 4,718,430; 9,713,600, 9,707,199, 9,687,461, 9,662,306, 9,561,206, U.S. Publ. No. 2011/0190702; U.S. Publ. No. 2017/0266144, U.S. Publ. No. 2016/0324814, U.S. Publ. No. 2016/0195559, U.S. Publ. No. 2016/0195558, U.S. Publ. No. 2016/0193172, 2 U.S. Publ. No. 016/0193171, U.S. Publ. No. 2016/0193170, WO 2016/111843, DE 2615061; and in conjunction with diagnostic devices, for example, as provided in U.S. Publ. No. 2012/0072236. The contents of each of the foregoing patent documents is incorporated herein by reference in its entirety.

### Diagnosis and Monitoring

Provided herein are methods for the diagnosis and monitoring of conditions provided herein.

In some embodiments, the method of diagnosis or monitoring may comprise the step of measuring a percentage of pentadecanoylcarnitine as described herein, in a bodily fluid. In some embodiments, the method of diagnosis or monitoring may comprise the step of measuring a marker of a condition provided herein in a subject. In some embodiments, a correlation between one marker and another may prove instructive. In some embodiments, a condition provided herein may be diagnosed by reference to a threshold level of a marker of the condition, for example, serum pentadecanoylcarnitine percentage, serum concentration of pentadecanoylcarnitine, serum total pentadecanoylcarnitine and pentadecanoic acid, or a ratio between pentadecanoylcarnitine and pentadecanoic acid. For example, the threshold may be determined by reference to a symptom or marker of a condition provided herein.

The percentage of pentadecanoylcarnitine, or a marker of a condition provided herein in a subject may be monitored by any means. Samples for analysis may be derived any fluid or tissue of the subject. For example, from serum, plasma, erythrocyte membranes, urine, and feces.

### Example 1

A series of studies were conducted to identify additional serum-based compounds in dolphins, beyond pentadecanoic acid, which were associated with a lower risk of chronic disease indices. Promising compounds were then tested at a variety of concentrations for direct, clinically relevant activities that are known to prevent, manage, treat or cure chronic conditions.

Among bottlenose dolphins fed a modified fish diet containing higher concentrations of C15:0, it was hypothesized that downstream C15:0 metabolites may be contributing to the observed health benefits in this case-control study (Venn-Watson, S., Baird, M., Novick, B., Parry, C., Jensen, E. D. Modified fish diet shifted serum metabolome and alleviated chronic anemia in bottlenose dolphins (Tursiops truncatus): Potential role of odd-chain saturated fatty acids. PLoS ONE doi10.1371/journal.pone.0230769 (2020)). To test this hypothesis, changes in the dolphin serum metabolome during Months 0, 1, 3 and 6 were evaluated and compared between dolphins on the baseline and modified fish diet.

### Methods

Methods for this modified diet study, including metabolomics, were those previously described (Venn-Watson, S., Baird, M., Novick, B., Parry, C., Jensen, E. D. Modified fish diet shifted serum metabolome and alleviated chronic anemia in bottlenose dolphins (Tursiops truncatus): Potential role of odd-chain saturated fatty acids. PLoS ONE doi10.1371/journal.pone.0230769 (2020)), including bioinformatics, principal component analysis, hierarchical clustering, and random forest regression to identify metabolites that best predicted dolphins on the modified versus baseline diet.

### Bioinformatics

The informatics system consisted of four major components, the Laboratory Information Management System (LIMS), the data extraction and peak-identification software, data processing tools for QC and compound identification, and a collection of information interpretation and visualization tools for use by data analysts. The hardware and software foundations for these informatics components were the LAN backbone, and a database server running Oracle 10.2.0.1 Enterprise Edition. Peaks were quantified using area-under-the-curve. For studies spanning multiple days, a data normalization step was performed to correct variation resulting from instrument inter-day tuning differences. Essentially, each compound was corrected in run-day blocks by registering the medians to equal one (1.00) and normalizing each data point proportionately (termed the “block correction”). For studies that did not require more than one day of analysis, no normalization is necessary, other than for purposes of data visualization. In certain instances, biochemical data may have been normalized to an additional factor (e.g., cell counts, total protein as determined by Bradford assay, osmolality, etc.) to account for differences in metabolite levels due to differences in the amount of material present in each sample. Two-way ANOVA main effects models, including study group, month, and sex, were used to determine primary drivers of differences in the metabolome.

### Principal Components Analysis and Hierarchical Clustering

Each principal component was a linear combination of every metabolite and the principal components were uncorrelated. The number of principal components was equal to the number of observations. The first principal component was computed by determining the coefficients of the metabolites that maximized the variance of the linear combination. The second component found the coefficients that maximize the variance with the condition that the second component was orthogonal to the first. The third component was orthogonal to the first two components and so on. The total variance was defined as the sum of the variances of the predicted values of each component (the variance is the square of the standard deviation), and for each component, the proportion of the total variance was computed. Hierarchical clustering was used as an unsupervised method for clustering the data to show any large-scale differences. Complete clustering using the Euclidean distance was applied, where each sample was a vector with all metabolite values.

### Random Forest Regression

Random forest, a supervised classification technique based on an ensemble of decision trees, was used to provide “importance” rank ordering of serum biochemicals that changed due to the modified diet. A random subset of the data with identifying true class information was selected to build the tree (“bootstrap sample” or “training set”), and then the remaining data, the “out-of-bag” (OOB) variables, were passed down the tree to obtain a class prediction for each sample. This process was repeated thousands of times to produce the forest. The final classification of each sample was determined by computing the class prediction frequency (“votes”) for the OOB variables over the whole forest. This method was unbiased since the prediction for each sample was based on trees built from a subset of samples that did not include that sample. When the full forest was grown, the class predictions were compared to the true classes, generating the “OOB error rate” as a measure of prediction accuracy. Thus, the prediction accuracy was an unbiased estimate of how well one can predict sample class in a new data set. To determine which biochemicals made the largest contribution to the classification, a “variable importance” measure was computed. The “Mean Decrease Accuracy” (MDA) was used as this metric. The MDA was determined by randomly permuting a variable, running the observed values through the trees, and then reassessing the prediction accuracy.

### Results

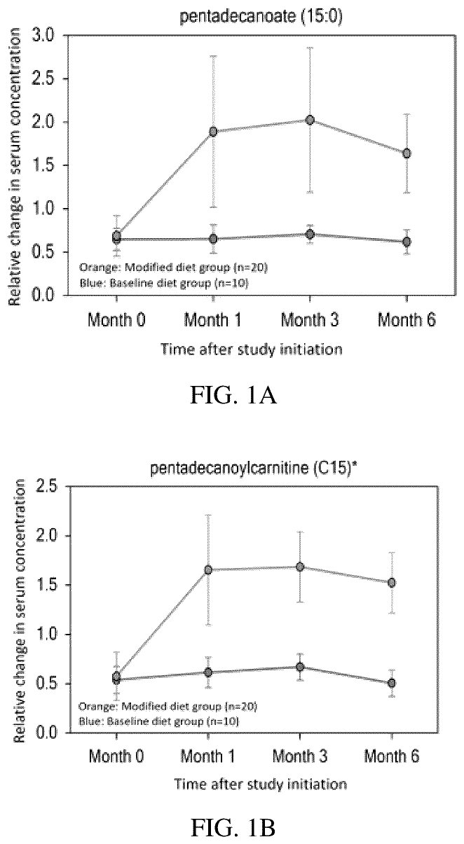

FIG. 1A shows increased serum pentadecanoic acid (C15:0) and FIG. 1B shows increased pentadecanoylcarnitine concentrations within 1 month on the modified fish diet that was sustained through 6 months. While there were no significant differences in serum C15:0 or pentadecanoylcarnitine concentrations between case and control groups at baseline (Month 0) (p=0.81 for C15:0 and p=0.86 for pentadecanoylcarnitine) using an ANOVA contrast model, both serum C15:0 and pentadecanoylcarnitine concentrations were higher among case versus control groups at Month 1 (p<0.0001 for C15:0 and p<0.0001 for pentadecanoylcarnitine), Month 3 (p<0.0001 for C15:0 and p<0.0001 for pentadecanoylcarnitine), and Month 6 (p<0.0001 for C15:0 and p<0.0001 for pentadecanoylcarnitine). As outlined in Table 1, both C15:0 (pentadecanoic acid) and pentadecanoylcarnitine were ranked within the top 30 important biochemical predictors of dolphins on the modified fish diet by the first month. Further, as the study moved from Month 1 to Month 6, pentadecanoylcarnitine's ranking increased to become the most important biochemical among dolphins on the modified diet.

TABLE 1

| | Ranking of biochemical importance in | | | |
|---|---|---|---|---|
| | predicting dolphins on the modified versus baseline diet | | | |
| Serum Biochemical | Month 0 | Month 1 | Month 3 | Month 6 |
| Pentadecanoic acid (C15:0) | 0 | 9^{th} | 5^{th} | 19^{th} |
| Pentadecanoylcamitine | 0 | 25^{th} | 2^{nd} | 1^{st} |

### Example 2

Given the observed increase in circulating concentrations of pentadecanoylcarnitine among dolphins on the modified fish diet, it was hypothesized that higher serum pentadecanoylcarnitine concentrations would correlate with changes in clinically relevant indices.

### Methods

Methods for this correlation study have been previously described (Venn-Watson, S., Baird, M., Novick, B., Parry, C., Jensen, E. D. Modified fish diet shifted serum metabolome and alleviated chronic anemia in bottlenose dolphins (Tursiops truncatus): Potential role of odd-chain saturated fatty acids. PLoS ONE doi10.1371/journal.pone.0230769 (2020)), including Pearson's correlations to evaluate correlations between relative changes in serum pentadecanoylcarnitine concentrations and log-transformed cholesterol, glucose, hemoglobin, red blood cell count, and platelets; and Spearman correlations for glomerular filtration rate and insulin.

### Results

Table 2 shows that rising serum pentadecanoylcarnitine concentrations observed in the modified diet study were correlated with lower cholesterol and lower insulin. Rising serum pentadecanoylcarnitine concentrations were correlated with higher hemoglobin, red blood cell count, platelets, and glomerular filtration rate. These data support that increasing serum pentadecanoylcarnitine concentrations may play a role in improving chronic conditions, including hypercholesterolemia, hyperinsulinemia, anemia, thrombocytopenia, and renal disease.

TABLE 2

| Tested Correlations with Serum | | |
|---|---|---|
| Pentadecanoylcarnitine Concentrations | Correlation | P-value |
| Cholesterol | −0.26 | 0.005 |
| Glucose | 0.02 | 0.87 |
| Hemoglobin | +0.45 | <0.0001 |
| Red blood cell count | +0.44 | <0.0001 |
| Platelets | +0.31 | 0.0006 |
| Glomerular filtration rate | +0.18 | 0.05 |
| Insulin | −0.24 | 0.008 |

### Example 3

Given our prior demonstration of C15:0's biologically relevant activities in human cell systems mimicking various chronic disease states, including lowering inflammation and fibrosis (Venn-Watson, S., Lumpkin, R., Dennis, E. A. Efficacy of dietary odd-chain saturated fatty acid pentadecanoic acid parallels broad associated health benefits in humans: could it be essential?Sci Rep 10:8161 (2020)), it was hypothesized that pentadecanoylcarnitine would have similar biologically relevant activities.

### Methods

Methods for this human primary cell phenotypic profiling study were those previously described (Venn-Watson, S., Lumpkin, R., Dennis, E. A. Efficacy of dietary odd-chain saturated fatty acid pentadecanoic acid parallels broad associated health benefits in humans: could it be essential?Sci Rep 10:8161 (2020)). Significant activities of pentadecanoylcarnitine were those that were equal to or greater than 0.1 | log 10| at 6.7 μM. Biomarkers with significant changes caused by pentadecanoylcarnitine were compared with results from the same study using pentadecanoic acid (C15:0).

### Results

Table 3 demonstrates clear differences in primary cell-based activities between pentadecanoylcarnitine and pentadecanoic acid. Of 24 identified biological activities caused by pentadecanoylcarnitine, no specific biomarkers changed significantly in the same direction with pentadecanoic acid.

This study supports that pentadecanoylcarnitine has biologically activities relevant to preventing, managing or treating autoimmune diseases, allergies, asthma, chronic inflammation, cardiovascular disease, oncology, chronic obstructive pulmonary disease, lung inflammation, restenosis, fibrosis, dermatitis, psoriasis, and wound healing that are surprisingly distinct from pentadecanoic acid.

TABLE 3

| Cell | Disease | Representative | Pentadecanoic | | |
|---|---|---|---|---|---|
| System | Biomarker | Disease State | acid (C15:0) | | Pentadecanoylcarnitine |
| 4H | VEGFR2 | Autoimmunity, | NS | (0.03) | −0.11 |
| | | allergy, asthma | | | |
| LPS | Tissue Factor | Chronic inflammation, | NS | (0.03) | +0.11 |
| | | cardiovascular disease | | | |
| SAg | E-Selectin | Chronic inflammation, | NS | (−0.06) | −0.11 |
| | B-Cell | autoimmune disease | NS | (0.04) | −0.17 |
| | Proliferation | | | | |
| BT | sIL-6 | Asthma, oncology, | +0.12 | | −0.13 |
| | sTNF-α | autoimmunity, allergy | +0.14 | | −0.10 |
| BE3C | uPAR | COPD, lung | NS | (0.03) | −0.12 |
| | CXCL10/IP-10 | inflammation | NS | (−0.004) | −0.10 |
| | CXCL11/I-TAC | | NS | (−0.02) | −0.54 |
| | CXCL8/IL-8 | | NS | (−0.002) | −0.21 |
| | CXCL9/MIG | | NS | (0.001) | −0.60 |
| | HLA-DR | | NS | (0.008) | −0.63 |
| | IL-1a | | NS | (0.005) | −0.19 |
| | MMP-1 | | NS | (−0.02) | −0.27 |
| | MMP-9 | | NS | (0.01) | −0.34 |
| | PAI-1 | | NS | (0.01) | −0.24 |
| | tPA | | NS | (0.03) | −0.43 |
| | uPA | | NS | (−0.002) | −0.28 |
| CASM3C | Proliferation | Cardiovascular | NS | (−0.04) | +0.10 |
| | | inflammation, | | | |
| | | restonosis | | | |
| HDF3CGF | MMP-1 | Fibrosis, chronic | NS | (−0.03) | −0.12 |
| | | inflammation | | | |
| KF3CT | CXCL10/IP-10 | Dermatitis, psoriasis | NS | (0.006) | −0.10 |
| MyoF | α-SM Actin | Wound healing, matrix | NS | (0.02) | −0.26 |
| | | remodeling, fibrosis, | | | |
| | | chronic inflammation | | | |
| /IMphg | MCP-1 | Chronic inflammation, | NS | (0.009) | −0.25 |
| | sIL-10 | restenosis, cardiovascular | NS | (−0.02) | −0.13 |
| | | disease | | | |

### Example 4

Given demonstration of pentadecanoylcarnitine as a biologically active compound in human cell systems mimicking various disease states, it was hypothesized that pentadecanoylcarnitine would have receptor-based pharmacological activities.

### Methods

Methods for this study have been previously described (Venn-Watson, S., Lumpkin, R., Dennis, E. A. Efficacy of dietary odd-chain saturated fatty acid pentadecanoic acid parallels broad associated health benefits in humans: could it be essential?Sci Rep 10:8161 (2020)). Significant activities of pentadecanoylcarnitine were those tagged as having maximum pharmacological activities that were greater than or equal to 70% that of the internal control between the concentrations of 1 to 20 μM. Significant pharmacological activities caused by pentadecanoylcarnitine were compared with results from the same assays using pentadecanoic acid (C15:0).

### Results

Table 4 shows multiple pharmacological activities, at RC50 concentrations between approximately 3 and 10 μM, of pentadecanoylcarnitine, approximately matching maximum activities of positive controls.

Pentadecanoylcarnitine is a cannabinoid receptor 1 and receptor 2 agonist at approximately 1 to 20 μM, further supporting its role in preventing, managing and treating cardiovascular disease, cancer, inflammation, arthritis and cholestatic pruritis (as shown at 6.7 μM in Example 3). Compounds with these activities have also been used or proposed to be used to prevent, manage, or treat nausea, obesity, neuropathic pain with multiple sclerosis, pain, gastrointestinal disorders, atherosclerosis, symptoms of multiple sclerosis, spinal cord injury, Alzheimer's disease, and amyotrophic lateral sclerosis, tics and behavioral problems with Tourette's syndrome, anxiety disorders, attention-deficit hyperactivity disorder, depression, brain damage, tardive dyskinesia, glaucoma, and cough (Pertwee, R. G. Emerging strategies for exploiting cannabinoid receptor agonists as medicines. Br J Pharmacol 156:397-411 (2009), Bryk, M. and Starowicz, K. Cannabinoid-based therapy as a future for joint degeneration. Focus on the role of CB2 receptor in the arthritis progression and pain: an updated review. Pharmacol Rep 73:681-699 (2021)).

The two most potent endogenous cannabinoids (also called endocannabinoids) are anadamide and 2-arachidonoylglyerol (Battista, N., Tommaso, M. D., Bari, M., Maccarrone, M. The endocannabinoid system: an overview. Front Behav Neurosci doi 10.3389/fnbeh.2012.00009 (2012).

Bouchouriab, F. Z., Fortin, M., Noll, C., Dube, J., Carpentier, A. C. Plasma palmitoyl-carnitine (AC16:0) is a marker of increased postprandial nonesterified incomplete fatty acid oxidation rate in adults with type 2 diabetes. Can J Diabetes 42:382-388.el (2018)). These compounds bind to an activate both cannabinoid 1 (CB1) and cannabinoid 2 (CB2) receptors found throughout the body, as well as peroxisome proliferator-activated receptors (PPARs). Through these mechanisms, naturally occurring endocannabinoids in the body have demonstrated a wide array of health benefits, including those related to modulating memory, cancer, appetite, fertility, pain, obesity, nausea, osteoarthritis, cardiovascular disease, metabolic disorders, hepatic diseases, inflammation, and neurological and neuropsychiatric disorders (Ligresti, A., Petrosino, S. Di Marzo, V. From endocannabinoid profiling to ‘endocannabinoid therapeutics’. Curr Opin Chem Biol 13:21-31 (2009)).

Pentadecanoylcarnitine is a histamine H1 and H2 receptor antagonist at approximately 1 to 20 μM, further supporting its role in preventing, managing and treating allergies, asthma and dermatitis (as shown at 6.7 μM in Example 3). Compounds with these activities have also been used to manage or treat other hypersensitivity disorders and peptic ulcers.

Pentadecanoylcarnitine is a dopamine receptor D1 antagonist at approximately 1 to 20 μM. Compounds with these activities have been used or proposed to be used to prevent, manage, or treat neurological diseases, including Parkinson disease, as well as mental disorders, schizophrenia, cocaine abuse, obesity, pathological gambling, and Tourette's syndrome.

Pentadecanoylcarnitine is an α-2A adrenergic receptor agonist at approximately 1 to 20 μM, further supporting its role in preventing, managing and treating allergic reactions, asthma, nasal and sinus congestion, allergic rhinitis and facial erythema associated with rosacea (as shown at 6.7 μM in Example 3). Compounds with these activities have been used or proposed to be used to support weight loss.

Pentadecanoylcarnitine is a cholinergic receptor muscarinic 1 antagonist at approximately 1 to 20 μM, further supporting its role in preventing, managing and treating allergies, chronic obstructive pulmonary disease (COPD), and asthma (as shown at 6.7 μM in Example 3). Compounds with these activities have been used or proposed to be used to prevent, manage, or treat movement disorders including Parkinson disease, sleep disorders, nausea, inflammatory bowel syndrome spasms, mental disorders, peptic ulcers, hyperactive bladder, and depression.

Pentadecanoylcarnitine is a SERT blocker at approximately 1 to 20 PM. Compounds with these activities have been used or proposed to be used to prevent, manage, or treat depression and obsessive-compulsive disorders.

Pentadecanoylcarnitine is a 5-HT1A receptor agonist at approximately 1 to 20 μM. Compounds with these activities have been used or proposed to be used to regulate mood, sleep, nausea, sexuality and appetite, including increased sociability, decreased impulsivity, facilitation of sex drive and arousal, diminished food intake, and prolonged REM sleep. Compounds that activate this receptor have also been used as a means to prevent, manage, and treat depression, migraines, bipoloar disorder, anxiety, hypertension, pain, schizophrenia, Parkinson's disease, aggression, drug-seeking behavior, and opioid-induced respiratory depression.

Pentadecanoylcarnitine is a 5-HT1B receptor agonist at approximately 1 to 20 μM. Compounds with these activities have been used or proposed to be used to manage depression and anxiety, and to reduce of aggression and impulsivity.

In summary, this study has identified pentadecenoyl carnitine as a newly discovered endocannabinoid that fully activates both cannabinoid 1 and 2 receptors at concentrations between 1 and 20 μM.

TABLE 4

| | | Max Response | |
|---|---|---|---|
| | | (%) Compared to | |
| RC50 (μM) | | Positive Control | |
| | C15:0 | | C15:0 |

| | | | |
|---|---|---|---|
| Result | Acyl- | Acyl- | Positive |

| Assay Target | Activity | Type | C15:0 | carnitine | C15:0 | carnitine | Control | Medical uses |
|---|---|---|---|---|---|---|---|---|
| Cholinergic | Antagonist | IC50 | >20 | 10.5 | 0 | 77 | Atropine | Allergies, movement disorders |
| receptor | | | | | | | | (Parkinson disease), sleep, |
| muscarinic 1 | | | | | | | | nausea, IBS spasms, COPD, |
| | | | | | | | | asthma, antipsychotics, peptic |
| | | | | | | | | ulcers, overactive bladder, |
| | | | | | | | | antidepressant |
| Cholinergic | Agonist | EC50 | >20 | 3.9 | 2 | 206 | Acetyl- | Contracts smooth muscles, |
| receptor | | | | | | | choline | dilates blood vessels, and slows |
| muscarinic 2 | | | | | | | chloride | heart rate |
| Histamine H1 | Antagonist | IC50 | >20 | 10.9 | 5 | 71 | Mepyramine | Allergy, hypersensitivity |
| receptor | | | | | | | | reactions, pruritic skin disorders |
| Histamine H2 | Antagonist | IC50 | >20 | 6.1 | 2 | 98 | Tiotidine | Peptic ulcers |
| receptor | | | | | | | | |
| Cannabinoid | Agonist | EC50 | >20 | 3.7 | 25 | 111 | CP 55940 | Nausea, obesity, neuropathic |
| receptor 1 | | | | | | | | pain with multiple sclerosis |
| Cannabinoid | Agonist | EC50 | >20 | 3.2 | 22 | 106 | CP 55940 | pain, gastrointestinal disorders, |
| receptor 2 | | | | | | | | atherosclerosis, cardiovascular |
| | | | | | | | | disease, cancer, symptoms of |
| | | | | | | | | multiple sclerosis/spinal cord |
| | | | | | | | | injury/Alzheimer’s |
| | | | | | | | | disease/amyotrophic lateral |
| | | | | | | | | sclerosis, tics and behavioral |
| | | | | | | | | problems with Tourette’s |
| | | | | | | | | syndrome, anxiety disorders, |
| | | | | | | | | attention-deficit hyperactivity |
| | | | | | | | | disorder, depression, brain |
| | | | | | | | | damage, tardive dyskinesia, |
| | | | | | | | | glaucoma, cough, cholestatic |
| | | | | | | | | pruritis, arthritis, inflammation |
| | | | | | | | | (Pertwee, R.G. Emerging |
| | | | | | | | | strategies for exploiting |
| | | | | | | | | cannabinoid receptor agonists as |
| | | | | | | | | medicines. Br J Pharmacol |
| | | | | | | | | 156: 397-411 (2009), Bryk, M. |
| | | | | | | | | and Starowicz, K. Cannabinoid- |
| | | | | | | | | based therapy as a future for |
| | | | | | | | | joint degeneration. Focus on the |
| | | | | | | | | role of CB2 receptor in the |
| | | | | | | | | arthritis progression and pain: |
| | | | | | | | | an updated review. Pharmacol |
| | | | | | | | | Rep 73: 681-699 (2021)) |
| 5-HT1A | Agonist | EC50 | >20 | 9.6 | 0 | 78 | Serotonin | Regulation of mood, sleep, |
| receptor | | | | | | | hydrochloride | nausea, sexuality and appetite, |
| | | | | | | | | including increased |
| | | | | | | | | sociability, decreased |
| | | | | | | | | impulsivity, facilitation of sex |
| | | | | | | | | drive and arousal, diminished |
| | | | | | | | | food intake, and prolonged |
| | | | | | | | | REM sleep. Means to treat |
| | | | | | | | | depression, migraines, bipolar |
| | | | | | | | | disorder, anxiety, |
| | | | | | | | | hypertension, pain, |
| | | | | | | | | schizophrenia, Parkinson’s |
| | | | | | | | | disease, aggression, drug- |
| | | | | | | | | seeking behavior, and opioid- |
| | | | | | | | | induced respiratory |
| | | | | | | | | depression. |
| 5-HT1B | Agonist | EC50 | >20 | 9.3 | 0 | 74 | Serotonin | Means to manage depression |
| receptor | | | | | | | hydrochloride | and anxiety, reduction of |
| | | | | | | | | aggression and impulsivity |
| α-2A | Agonist | EC50 | >20 | 5.9 | 22 | 105 | UK 14304 | Hypotension, allergic |
| adrenergic | | | | | | | | reactions, asthma, nasal and |
| receptor | | | | | | | | sinus congestion, allergic |
| | | | | | | | | rhinitis, facial erythema |
| | | | | | | | | associated with rosacea, |
| | | | | | | | | obesity |
| Dopamine | Antagonist | IC50 | >20 | 6.2 | 4 | 105 | SCH 39166 | Antipsychotic, schizophrenia, |
| receptor D1 | | | | | | | | cocaine abuse, obesity, |
| | | | | | | | | pathological gambling, |
| | | | | | | | | Tourette’s syndrome |
| Serotonin | Blocker | IC50 | >20 | 7.4 | 1 | 83 | Clomipramine | Antidepressant, obsessive- |
| Transporter | | | | | | | | compulsive disorder |

Various literature references include teachings related to fatty acid supplementation, including but not limited to Greenberg J A, Bell S J, Ausdal W V. Omega-3 Fatty Acid supplementation during pregnancy. Reviews in obstetrics & gynecology 2008, 1:162-169; Mihalik, S. J., Goodpaster, B. H., Kelley, D. E., Chace, D. H., Vockley, J., Toledo, F. G. S. et al. Increased levels of plasma acylcarnitines in obesity and type 2 diabetes and identification of a marker of glucolipotoxicity. Obesity 18:1695-1700 (2010); Venn-Watson, S., Parry, C., Baird, M., Stevenson, S., Carlin, K., Daniels, R, et al. Increased dietary intake of saturated fatty acid heptadecanoic acid (C17:0) associated with decreasing ferritin and alleviated metabolic syndrome in dolphins. PLoS ONE doi10.1371 (2015); Venn-Watson, S., Smith, C. R., Stevenson, S., Parry, C., Daniels, R. Jensen, E., et al. Blood-based indicators of insulin resistance and metabolic syndrome in bottlenose dolphins (Tursiops truncatus). Front Endocrinol:10.3389 (2013); Venn-Watson, S., Benham, C., Carlin, K., DeRienzo, D., St. Leger, J. Hemochromatosis and fatty liver disease: building evidence for insulin resistance in bottlenose dolphins (Tursiops truncatus).J Zoo Wildlf Med 43:10.1638 (2012); Venn-Watson, S., Smith, C. R., Gomez, F., Jensen, E. D. Physiology of aging among healthy, older bottlenose dolphins (Tursiops truncatus): comparisons with aging humans. J Comp Physiol 181:667-680 (2011).

### Exemplary Compositions, Uses, and Methods

Composition 1: A composition for treatment, management, amelioration, or prophylaxis of a condition selected from the group consisting of aggression, allergies, allergic rhinitis, Alzheimer's disease, anxiety, anxiety disorders, amyotrophic lateral sclerosis, arthritis, asthma, atherosclerosis, attention-deficit hyperactivity disorder, bipoloar disorder, brain damage, cancer, cardiovascular disease, cholestatic pruritis, depression, chronic obstructive pulmonary disease (COPD), cocaine abuse, cough, dermatitis, depression, drug-seeking behavior, gastrointestinal disorders, facial erythema associated with rosacea, glaucoma, hepatic diseases, hyperactive bladder, hypersensitivity disorders, hypertension, impulsivity, inflammation, mental disorders, mental conditions, metabolic disorders, migraines, nasal congestion, sinus congestion, nausea, neuropathic pain with multiple sclerosis, symptoms of multiple sclerosis, neurological disorders, neuropsychiatric disorders, obesity, obsessive-compulsive disorders, opioid-induced respiratory depression, osteoarthritis, pain, Parkinson disease, pathological gambling, peptic ulcers, schizophrenia, sleep disorders, spinal cord injury, tardive dyskinesia, tics with Tourette's syndrome, behavioral problems with Tourette's syndrome, the composition comprising: pentadecanoylcarnitine or pentadecanoic acid, or pharmaceutically acceptable salts, solvates, or stereoisomers thereof.

Composition 2: A composition for supporting appetite, cardiovascular health, hematologic health, memory, metabolic health, mood, prolonged REM sleep, renal health, sexuality, sociability, metabolic health, hematological health, renal health, and weight loss, the composition comprising: pentadecanoylcarnitine or pentadecanoic acid, or pharmaceutically acceptable salts, solvates, or stereoisomers thereof.

Composition 3: Composition 1 or 2, comprising pentadecanoylcarnitine.

Composition 4: Composition 1 or 2, comprising pentadecanoic acid.

Composition 5: Composition 1 or 2, comprising pentadecanoylcarnitine and pentadecanoic acid.

Composition 6: Any one of Compositions 1-5, configured for administration of from 0.1 mg to 50 mg, per 1 kg of body weight, optionally from 0.3 mg to 5 mg, per 1 kg of body weight, of the pentadecanoylcarnitine or pentadecanoic acid, or pharmaceutically acceptable salts, solvates, or stereoisomers thereof to a subject in need thereof.

Composition 7: Any one of Compositions 1-6, configured for administration once per day.

Composition 8: Any one of Compositions 1-7, in a unit dosage form comprising from 0.01 mg to 10000 mg, optionally from 10 mg to 200 mg, of the pentadecanoylcarnitine or pentadecanoic acid, or pharmaceutically acceptable salts, solvates, stereoisomers, or esters thereof.

Composition 9: Any one of Compositions 1-7, wherein the composition is in a form selected from the group consisting of a foodstuff, a dietary supplement, a unit dosage form, a prescription drug, or a pharmaceutical drug.

Composition 10: Any one of Compositions 1-7, wherein the composition is in a form selected from the group consisting of a dietary supplement, a medical food, a food additive, a food fortifier, a beverage additive, a beverage fortifier, a fortified food, a fortified beverage, an additized food, and an additized beverage.

Use 11: Use of any one of Compositions 1-10, in the manufacture of a medicament for achieving a body concentration of pentadecanoylcarnitine of from 1 μM to 20 μM.

Method 12: A method of treatment, management, amelioration, or prophylaxis of a condition selected from the group consisting of aggression, allergies, allergic rhinitis, Alzheimer's disease, anxiety, anxiety disorders, amyotrophic lateral sclerosis, arthritis, asthma, atherosclerosis, attention-deficit hyperactivity disorder, bipoloar disorder, brain damage, cancer, cardiovascular disease, cholestatic pruritis, depression, chronic obstructive pulmonary disease (COPD), cocaine abuse, cough, dermatitis, depression, drug-seeking behavior, gastrointestinal disorders, facial erythema associated with rosacea, glaucoma, hepatic diseases, hyperactive bladder, hypersensitivity disorders, hypertension, impulsivity, inflammation, mental disorders, mental conditions, metabolic disorders, migraines, nasal congestion, sinus congestion, nausea, neuropathic pain with multiple sclerosis, symptoms of multiple sclerosis, neurological disorders, neuropsychiatric disorders, obesity, obsessive-compulsive disorders, opioid-induced respiratory depression, osteoarthritis, pain, Parkinson disease, pathological gambling, peptic ulcers, schizophrenia, sleep disorders, spinal cord injury, tardive dyskinesia, tics with Tourette's syndrome, behavioral problems with Tourette's syndrome, the method comprising: administering to a subject in need thereof, an effective amount of pentadecanoylcarnitine or pentadecanoic acid, or pharmaceutically acceptable salts, solvates, or stereoisomers thereof.

Method 13: A method of supporting appetite, cardiovascular health, hematologic health, memory, metabolic health, mood, prolonged REM sleep, renal health, sexuality, sociability, metabolic health, hematological health, renal health, and weight loss, the composition comprising: administering to a subject in need thereof, an effective amount of pentadecanoylcarnitine or pentadecanoic acid, or pharmaceutically acceptable salts, solvates, or stereoisomers thereof.

Method 14: Method 12 or 13, wherein a body concentration of pentadecanoylcarnitine of from 1 μM to 20 μM is achieved in the subject.

Method 15: Method 12-14, wherein pentadecanoylcarnitine is administered.

Method 16: Method 12-14, wherein pentadecanoic acid is administered.

Method 17: Method 12-14, wherein pentadecanoylcarnitine and pentadecanoic acid are administered.

Method 18: Method 12-17, wherein from 0.1 mg to 50 mg, per 1 kg of body weight, optionally from 0.3 mg to 5 mg, per 1 kg of body weight, of the pentadecanoylcarnitine or pentadecanoic acid, or pharmaceutically acceptable salts, solvates, or stereoisomers thereof is administered.

Method 19: Method 12-18, wherein pentadecanoylcarnitine or pentadecanoic acid, or pharmaceutically acceptable salts, solvates, or stereoisomers thereof is administered once per day.

Method 20: Method 12-19, wherein pentadecanoylcarnitine or pentadecanoic acid, or pharmaceutically acceptable salts, solvates, or stereoisomers thereof is in a unit dosage form comprising from 0.01 mg to 10000 mg, optionally from 10 mg to 200 mg, of the pentadecanoylcarnitine or pentadecanoic acid, or pharmaceutically acceptable salts, solvates, stereoisomers, or esters thereof.

Method 21: Method 12-20, wherein pentadecanoylcarnitine or pentadecanoic acid, or pharmaceutically acceptable salts, solvates, or stereoisomers thereof is administered in a form selected from the group consisting of a foodstuff, a dietary supplement, a unit dosage form, a prescription drug, or a pharmaceutical drug.

Method 22: Method 12-20, wherein pentadecanoylcarnitine or pentadecanoic acid, or pharmaceutically acceptable salts, solvates, or stereoisomers thereof is administered in a form selected from the group consisting of a dietary supplement, a medical food, a food additive, a food fortifier, a beverage additive, a beverage fortifier, a fortified food, a fortified beverage, an additized food, and an additized beverage.

Dietary Supplement 23: A dietary supplement comprising pentadecanoylcarnitine, or pharmaceutically acceptable salts, solvates, or stereoisomers thereof.

Dietary Supplement 24: Dietary Supplement 23, configured for administration of a minimum of 0.1 mg per 1 kg of body weight, optionally of a minimum of 0.3 mg per 1 kg of body weight, optionally of a minimum of 1 mg per 1 kg of body weight, of the pentadecanoylcarnitine, or pharmaceutically acceptable salts, solvates, or stereoisomers thereof to a subject in need thereof.

Dietary Supplement 25: Any one of Dietary Supplements 23-24, configured for administration of from 0.1 mg to 50 mg, per 1 kg of body weight, optionally from 0.3 mg to 5 mg, per 1 kg of body weight, of the pentadecanoylcarnitine, or pharmaceutically acceptable salts, solvates, or stereoisomers thereof to a subject in need thereof.

Dietary Supplement 26: Any one of Dietary Supplements 23-25, configured for administration once per day.

Dietary Supplement 27: Any one of Dietary Supplements 23-26, in a unit dosage form comprising from 0.01 mg to 10000 mg, optionally from 1 mg to 1000 mg, optionally from 10 mg to 200 mg, of the pentadecanoylcarnitine, or pharmaceutically acceptable salts, solvates, or stereoisomers thereof.

Dietary Supplement 28: Any one of Dietary Supplements 23-27, in a form of a pharmaceutical drug or prescription drug.

Dietary Supplement 29: Any one of Dietary Supplements 23-27, in a form selected from the group consisting of a foodstuff, a medical food, a food additive, a food fortifier, a beverage additive, a beverage fortifier, a fortified food, a fortified beverage, an additized food, and an additized beverage.

Dietary Supplement 30: Any one of Dietary Supplements 23-27, in a form of a foodstuff.

Dietary Supplement 31: Any one of Dietary Supplements 23-27, in a form of a medical food.

Dietary Supplement 32: Any one of Dietary Supplements 23-27, in a form of a food additive.

Dietary Supplement 33: Any one of Dietary Supplements 23-27, in a form of a food fortifier.

Dietary Supplement 34: Any one of Dietary Supplements 23-27, in a form of a beverage additive.

Dietary Supplement 35: Any one of Dietary Supplements 23-27, in a form of a beverage fortifier.

Dietary Supplement 36: Any one of Dietary Supplements 23-27, in a form of a fortified food.

Dietary Supplement 37: Any one of Dietary Supplements 23-27, in a form of a fortified beverage.

Dietary Supplement 38: Any one of Dietary Supplements 23-27, in a form of an additized food.

Dietary Supplement 39: Any one of Dietary Supplements 23-27, in a form of an additized beverage.

Dietary Supplement 40: Any one of Dietary Supplements 23-39, for veterinary use.

Dietary Supplement 41: Any one of Dietary Supplements 23-39, for human use.

Composition 42: A composition substantially as described herein.

Method 43: A method substantially as described herein.

Use 44: A use substantially as described herein.

While the disclosure has been illustrated and described in detail in the drawings and foregoing description, such illustration and description are to be considered illustrative or exemplary and not restrictive. The disclosure is not limited to the disclosed embodiments. Variations to the disclosed embodiments can be understood and effected by those skilled in the art in practicing the claimed disclosure, from a study of the drawings, the disclosure and the appended claims.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

As used in the claims below and throughout this disclosure, by the phrase “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

1-20. (canceled)

21. A method of (1) supporting memory, sociability, prolonged REM sleep, or sexuality in a subject in need thereof; (2) treating, managing, ameliorating, or preventing a condition selected from the group consisting of: tics and behavioral problems with Tourette's syndrome, anxiety disorders, attention-deficit hyperactivity disorder, brain damage, tardive dyskinesia, glaucoma, cholestatic pruritis, hypotension, nasal and sinus congestion, allergic rhinitis, facial erythema associated with rosacea, antipsychotic, schizophrenia, cocaine abuse, pathological gambling, Tourette's syndrome in a subject in need thereof, the method comprising:
administering to the subject, an effective amount of pentadecanoic acid, or a pharmaceutically acceptable salt thereof.

22. The method of claim 21, wherein the method supports memory, sociability, prolonged REM sleep, or sexuality in the subject.

23. The method of claim 22, wherein the pentadecanoic acid, or a pharmaceutically acceptable salt thereof, is administered to the subject in an amount that modulates CB1 and CB2 receptors.

24. The method of claim 21, wherein the condition is tics and behavioral problems with Tourette's syndrome, anxiety disorders, attention-deficit hyperactivity disorder, brain damage, tardive dyskinesia, glaucoma, or cholestatic pruritis.

25. The method of claim 24, wherein pentadecanoic acid, or a pharmaceutically acceptable salt thereof, is administered to the subject in an amount that modulates CB1 and CB2 receptors.

26. The method of claim 21, wherein the condition is selected from the group consisting of: hypotension, nasal and sinus congestion, allergic rhinitis, and facial erythema associated with rosacea.

27. The method of claim 26, wherein pentadecanoic acid, or a pharmaceutically acceptable salt thereof, is administered to the subject in an amount that modulates alpha-2A adrenergic receptor.

28. The method of claim 21, wherein pentadecanoic acid, or a pharmaceutically acceptable salt thereof, is administered to the subject in an amount that modulates dopamine receptor D1.

29. The method of claim 28, wherein the condition is selected from the group consisting of: antipsychotic, schizophrenia, cocaine abuse, pathological gambling, and Tourette's syndrome.

30. The method of claim 21, wherein a body concentration of pentadecanoic acid of from 1 μM to 20 μM is achieved in the subject.

31. The method of claim 21, wherein from 2 mg to 50 mg, per 1 kg of body weight of the pentadecanoic acid, or a pharmaceutically acceptable salt thereof, is administered.

32. The method of claim 21, wherein the pentadecanoic acid, or a pharmaceutically acceptable salt thereof, is administered once per day.

33. The method of claim 21, wherein the pentadecanoic acid, or a pharmaceutically acceptable salt thereof, is in a unit dosage form comprising from 0.01 mg to 10000 mg of the pentadecanoic acid, or a pharmaceutically acceptable salt thereof.

34. The method of claim 21, wherein the pentadecanoic acid is in the form of a pharmaceutical drug or a prescription drug.

35. The method of claim 21, wherein the pentadecanoic acid is in a form selected from the group consisting of a foodstuff, a medical food, a food additive, a food fortifier, a beverage additive, a beverage fortifier, a fortified food, a fortified beverage, an additized food, and an additized beverage.

36. The method of claim 21, wherein the pentadecanoic acid, or a pharmaceutically acceptable salt thereof, is administered as a dietary supplement.

37. The method of claim 21, wherein the pentadecanoic acid, or a pharmaceutically acceptable salt thereof, is administered in a unit dosage form.

38. The method of claim 21, wherein the pentadecanoic acid, or a pharmaceutically acceptable salt thereof, is administered in a tablet, a capsule, an encapsulated pill, a gelcap pill, a dispensable form of an oral or injectable liquid suspension or solution, a spray, an aerosol, powder, or granules.

39. The method of claim 21, wherein the pentadecanoic acid, or a pharmaceutically acceptable salt thereof, is isolated from natural sources, semi-synthetic, synthetic, or any mixture thereof.

40. The method of claim 21, wherein the pentadecanoic acid, or a pharmaceutically acceptable salt thereof, is administered in a tablet or a capsule.
